(19) United States
(12) Patent Application Publication
(10) Pub. No.: US 2010/0209915 A1
(43) Pub. Date: Aug. 19, 2010

(54) Gene Expression Profiling for Identification, Monitoring, and Treatment of Ocular Disease

(76) Inventors: Danute M. Bankaitis-Davis, Boulder, CO (US); Lisa Siconolfi, Westminster, CO (US); Kathleen Storm, Longmont, CO (US); Karl Wassmann, Dover, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/520,675

(22) PCT Filed: Dec. 18, 2007
(86) PCT No.: PCT/US2007/025865

§ 371 Date: Apr. 1, 2010

Related U.S. Application Data
(60) Provisional application No. 60/876,098, filed on Dec. 19, 2006.

(51) Int. Cl. C12Q 1/68

(52) U.S. Cl. 435/6

(57) ABSTRACT

A method is provided in various embodiments for determining a profile data set for a subject with ocular disease or conditions related to ocular disease based on a sample from the subject, wherein the sample provides a source of RNAs. The method includes using amplification for measuring the amount of RNA corresponding to at least one constituent from Tables 1-5, 7-9, and 11-13. The profile data set comprises the measure of each constituent, and amplification is performed under measurement conditions that are substantially repeatable.

### REFERENCE TO RELATED APPLICATIONS

This application claims the benefit of U.S. Provisional Application No. 60/876,098 filed Dec. 19, 2006, the contents of which are incorporated by reference in its entirety.

### FIELD OF THE INVENTION

The present invention relates generally to the identification of biological markers associated with the identification of ocular disease. More specifically, the present invention relates to the use of gene expression data in the identification, monitoring and treatment of ocular disease and in the characterization and evaluation of conditions induced by or related to ocular disease.

### BACKGROUND OF THE INVENTION

Two leading causes of vision loss are glaucoma and age-related maculodegenerative disease (AMD). Glaucoma generally describes a group of diseases that damage the optic nerve, which transmits images from the light-sensitive inner back of the eye (retina) to the brain for interpretation. Because the optic nerve is unlikely to self repair, damage tends to be permanent and blindness can result. Glaucoma is a proliferative disease of the eye affecting 2.2 million patients in the U.S. and 65 million patients worldwide. It is related to the production and removal of the fluid in the eye known as the aqueous humor, a transparent fluid that provides nutrition to the lens and cornea and transmits light rays to the retina at the back of the eye. Aqueous humor leaves the eye through a sieve-like tissue called the trabecular meshwork, and glaucoma is believed to be caused by changes in the meshwork that prevent aqueous humor from leaving the eye. In the past, glaucoma was thought almost always to be related to high intraocular pressure that can result from problems such as a blocked fluid drainage system within the eye. However, evidence increasingly has shown that glaucoma can occur even when high intraocular pressure is absent.

There are several types of glaucoma, including primary open angle glaucoma (POAG), normal pressure glaucoma (NPG), and Pseudoexfoliative Glaucoma (PEX). POAG is the most common type of glaucoma often related to high intraocular pressure and the second leading cause of irreversible blindness in the United States. It is generally characterized by a clinical triad: (1) elevated intraocular pressure; (2) development of optic nerve atrophy; and (3) loss of peripheral field of vision, ultimately impairing central vision. The condition usually develops because the eye's drainage system functions improperly, sometimes due to blockages or constrictions that slowly cause fluid build-up. The term, open angle, is used with this type of glaucoma because the angle of the chamber where fluids build up to exit the eye is normal and not constricted.

NPG is a form of open angle glaucoma in which high intraocular pressure is absent. With NPG, vision loss tends to occur centrally rather than along the edges of the field of view, as with POAG. With PEX, a white, fiber-like material is deposited within the eye which can lead to blockages of the eye's drainage system, causing high intraocular pressure and damage to the optic nerve characteristic of open angle glaucoma. Reasons for formation of these types of deposits are unclear.

Age-related Maculodegenerative Disease (AMD) is a degenerative condition of the macula. It is the most common cause of vision loss in the United States in those 50 years old or older, and its prevalence increases with age. AMD is a major cause of visual impairment in the United States. Approximately 1.8 million Americans age 40 and older have advanced AMD, and another 7.3 million people with intermediate AMD are at substantial risk for vision loss. AMD is caused by hardening of the arteries that nourish the retina. This deprives the retinal tissue of oxygen and nutrients that it needs to function and thrive. As a result, the central vision deteriorates. AMD is classified as either wet (neovascular) or dry (non-neovascular), based on the absence or the presence of abnormal growth of blood vessels under the retina.

Wet AMD affects about 10% of patients who suffer from macular degeneration. This type occurs when new vessels form to improve the blood supply to oxygen-deprived retinal tissue. However, the new vessels are very delicate and break easily, causing bleeding and damage to surrounding tissue. The wet form can manifest in two types: classic or occult. Over 70% of patients with the wet form have the occult type. To date, only the classic wet type is treated with conventional laser photocoagulation to stabilize vision or to limit the growth of abnormal blood vessels. The remaining majority of patients with wet AMD cannot be treated with the laser procedure. The current laser treatment does not improve vision in most treated eyes because the laser destroys not only the abnormal blood vessel but also the overlying macula.

Dry AMD although more common, typically results in a less severe, more gradual loss of vision. It is characterized by drusen and loss of pigment in the retina. Drusen are small, yellowish deposits that form within the layers of the retina. The loss of vision associated with dry AMD tends to be milder and the disease progression is rather slow. There is no currently proven medical therapy for dry macular degeneration.

Glaucoma particularly is sight-threatening because, the disease often is difficult to detect in early stages due to a lack of symptoms, such as pain. In fact, glaucoma often is diagnosed only after vision already has been lost from optic nerve damage. Symptoms that do present can typically include gradual deterioration of vision, particularly loss of peripheral vision, creating tunnel vision and eventual blindness.

AMD also produces a slow loss of vision. Like glaucoma, both wet and dry AMD is difficult to detect in early stages due to lack of initial symptoms. Early signs of vision loss associated with AMD can include seeing shadowy areas in your central vision or experiencing unusually fuzzy or distorted vision. The dry form of macular degeneration will initially often cause slightly blurred vision. The center of vision may then become blurred and this region grows larger as the disease progresses. No symptoms may be noticed if only one eye is affected. In wet macular degeneration, straight lines may appear wavy and central vision loss can occur rapidly.

Since individuals with glaucoma and AMD can live for several years asymptomatic while the disease progresses, regular screenings are essential to detect these diseases at an early stage. Early detection of ocular disease preserves vision longer and makes the disease more manageable without invasive procedures. Thus a need exists for better ways to diagnose and monitor the progression and treatment of ocular disease.

Additionally, information on any condition of a particular patient and a patient's response to types and dosages of therapeutic or nutritional agents has become an important issue in clinical medicine today not only from the aspect of efficiency of medical practice for the health care industry but for improved outcomes and benefits for the patients. Thus, there is the need for tests which can aid in the diagnosis and monitor the progression and treatment of ocular disease.

### SUMMARY OF THE INVENTION

The invention is in based in part upon the identification of gene expression profiles (Precision Profiles™) associated with ocular disease. These genes are referred to herein as ocular disease associated genes. More specifically, the invention is based upon the surprising discovery that detection of as few as two ocular disease associated genes in a subject derived sample is capable of identifying individuals with or without ocular disease with at least 75% accuracy. More particularly, the invention is based upon the surprising discovery that the methods provided by the invention are capable of detecting ocular disease by assaying blood samples.

In various aspects the invention provides methods of evaluating the presence or absence (e.g., diagnosing or prognosing) of ocular disease, based on a sample from the subject, the sample providing a source of RNAs, and determining a quantitative measure of the amount of at least one constituent of any constituent (e.g., ocular disease associated gene) of any of Tables 1-5, 7-9, and 11-13, and arriving at a measure of each constituent. In a particular embodiment, the invention provides a method for evaluating the presence of ocular disease in a subject based on a sample from the subject, the sample providing a source of RNAs, comprising: a) determining a quantitative measure of the amount of at least one constituent of any constituent of any one table selected from the group consisting of Table 1A, Table 1B and Table 2 as a distinct RNA constituent in the subject sample, wherein such measure is obtained under measurement conditions that are substantially repeatable and the constituent is selected so that measurement of the constituent distinguishes between a normal subject and an ocular disease-diagnosed subject in a reference population with at least 75% accuracy; and b) comparing the quantitative measure of the constituent in the subject sample to a reference value.

Also provided by the invention is a method for assessing or monitoring the response to therapy (e.g., individuals who will respond to a particular therapy (“responders), individuals who won't respond to a particular therapy (“non-responders”), and/or individuals in which toxicity of a particular therapeutic may be an issue), in a subject having ocular disease or a condition related to ocular disease, based on a sample from the subject, the sample providing a source of RNAs, the method comprising: i) determining a quantitative measure of the amount of at least one constituent of any panel of constituents in Tables 1-5, 7-9, and 11-13 as a distinct RNA constituent, wherein such measure is obtained under measurement conditions that are substantially repeatable to produce a patient data set; and ii) comparing the patient data set to a baseline profile data set, wherein the baseline profile data set is related to the ocular disease, or conditions related to ocular disease.

In a further aspect, the invention provides a method for monitoring the progression of ocular disease or a condition related to ocular disease in a subject, based on a sample from the subject, the sample providing a source of RNAs, the method comprising: a) determining a quantitative measure of the amount of at least one constituent of any constituent of Tables 1-5, 7-9, and 11-13 as a distinct RNA constituent in a sample obtained at a first period of time to produce a first patient data set; and determining a quantitative measure of the amount of at least one constituent of any constituent of Tables 1-5, 7-9, and 11-13, as a distinct RNA constituent in a sample obtained at a second period of time to produce a second profile data set, wherein such measurements are obtained under measurement conditions that are substantially repeatable. Optionally, the constituents measured in the first sample are the same constituents measured in the second sample. The first subject data set and the second subject data set are compared allowing the progression of ocular disease in a subject to be determined. The second subject sample is taken e.g., one day, one week, one month, two months, three months, 1 year, 2 years, or more after first subject sample.

In various aspects the invention provides a method for determining a profile data set, i.e., an ocular disease profile, for characterizing a subject with ocular disease or conditions related to ocular disease based on a sample from the subject, the sample providing a source of RNAs, by using amplification for measuring the amount of RNA in a panel of constituents including at least one constituent from any of Tables 1-5, 7-9, and 11-13, and arriving at a measure of each constituent. The profile data set contains the measure of each constituent of the panel.

Also provided by the invention is a method of characterizing ocular disease or conditions related to ocular disease in a subject, based on a sample from the subject, the sample providing a source of RNAs, by assessing a profile data set of a plurality of members, each member being a quantitative measure of the amount of a distinct RNA constituent in a panel of constituents selected so that measurement of the constituents enables characterization of ocular disease.

In yet another aspect the invention provides a method of characterizing ocular disease or conditions related to ocular disease in a subject, based on a sample from the subject, the sample providing a source of RNAs, by determining a quantitative measure of the amount of at least one constituent from Tables 1-5, 7-9, and 11-13.

Additionally, the invention includes a biomarker for predicting individual response to ocular disease treatment in a subject having ocular disease or a condition related to ocular disease comprising at least one constituent of any constituent of Tables 1-5, 7-9, and 11-13.

The methods of the invention further include comparing the quantitative measure of the constituent in the subject derived sample to a reference value or a baseline value, e.g. baseline data set. The reference value is for example an index value. Comparison of the subject measurements to a reference value allows for the present or absence of ocular disease to be determined, response to therapy to be monitored or the progression of ocular disease to be determined. For example, a similarity in the subject data set compared to a baseline data set derived from a subject having ocular disease indicates the presence of ocular disease or response to therapy that is not efficacious. Whereas a similarity in the subject data set compares to a baseline data set derived from a subject not having ocular disease indicates the absence of ocular disease or response to therapy that is efficacious. In various embodiments, the baseline data set is derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure, and the baseline profile data set may be derived from one or more other samples from one or more different subjects.

The baseline profile data set may be derived from one or more other samples from the same subject taken under circumstances different from those of the first sample, and the circumstances may be selected from the group consisting of (i) the time at which the first sample is taken (e.g., before, after, or during treatment for ocular disease), (ii) the site from which the first sample is taken, (iii) the biological condition of the subject when the first sample is taken.

The measure of the constituent is increased or decreased in the subject compared to the expression of the constituent in the reference, e.g., normal reference sample or baseline value. The measure is increased or decreased 10%, 25%, 50% compared to the reference level. Alternately, the measure is increased or decreased 1, 2, 5 or more fold compared to the reference level.

In various aspects of the invention the methods are carried out wherein the measurement to conditions are substantially repeatable, particularly within a degree of repeatability of better than ten percent, five percent or more particularly within a degree of repeatability of better than three percent, and/or wherein efficiencies of amplification for all constituents are substantially similar, more particularly wherein the efficiency of amplification is within ten percent, more particularly wherein the efficiency of amplification for all constituents is within five percent, and still more particularly wherein the efficiency of amplification for all constituents is within three percent or less.

In addition, the one or more different subjects may have in common with the subject at least one of age group, gender, ethnicity, geographic location, nutritional history, medical condition, clinical indicator, medication, physical activity, body mass, and environmental exposure. A clinical indicator may be used to assess ocular disease or condition related to ocular disease of the one or more different subjects, and may also include interpreting the calibrated profile data set in the context of at least one other clinical indicator, wherein the at least one other clinical indicator includes blood chemistry, molecular markers in the blood, fluourescein angiography, other chemical assays, and physical findings.

The panel of constituents are selected so as to distinguish from a normal and a ocular disease-diagnosed subject. Alternatively, the panel of constituents is selected as to permit characterizing the severity of ocular disease in relation to a normal subject over time so as to track movement toward normal as a result of successful therapy and away from normal in response to ocular disease recurrence. Thus, in some embodiments, the methods of the invention are used to determine efficacy of treatment of a particular subject.

Preferably, the panel of constituents are selected so as to distinguish, e.g., classify between a normal and a ocular disease-diagnosed subject with at least 75%, 80%, 85%, 90%, 95%, 97%, 98%, 99% or greater accuracy. By “accuracy” is meant that the method has the ability to distinguish, e.g., classify, between subjects having ocular disease or conditions associated with ocular disease, and those that do not. Accuracy is determined for example by comparing the results of the Gene Precision Profilind™ to standard accepted clinical methods of diagnosing ocular disease, e.g., one or more symptoms of ocular disease such as gradual deterioration of vision, loss of peripheral vision, tunnel vision, seeing shadowy areas in your central vision or experiencing unusually fuzzy or distorted vision, loss of central vision, straight lines appearing wavy, and blindness.

At least 1, 2, 3, 4, 5, 6, 7, 8, 9, 10, 15, 20, 25, 30, 40, 50, 60, or 70 or more constituents are measured. In one aspect, one or more constituents from Tables 1-5, 7-9, and 11-13 is measured. In a preferred embodiment, one or more constituents selected from TGFB1 and MMP19 is measured. In another aspect, two or more constituents from Tables 1-5, 7-9, and 11-13 is measured. Preferably, two or more constituents selected from TGFB1, CRP, MADD, MMP19, CASP9, MMP13, NFKB1B, JUN, BCL3, BCL2L1, BAX, CD69, CD44, VDAC1, NFKB1, TIMP3, CD4, NOS2A, TRAF2, BIRC3, MMP2, MAPK14, IL8, HSPA1A, BIK, MMP9, MMP3, MMP12, PDCD8, C1QA, NOS1, TIMP1, TNFSF12, BID, ECE1, IL1RN, TNFRSF1B, TGFα, CD68, SAA1, GSR, BAD, SERPINA3, BAK1, CD3Z, TRADD, MAPK1, PPARα, CASP3, TP53, TRAF3, MAP3K1, HLADRB1, SOD2, IFNG, PTGS2, PLAU, ANXA11, LTA, APAF1, CASP1, TOSO, CD19, MMP15, TNFRSF1A, BIRC2, GSTA1, PDCD8, and IVIMP1 is measured. Even more preferably, TGFB1 and one or more of the following: SERPINB2, and CD69; ii) MMP19; and iii) MMF19 and CD69 is measured.

In some embodiments, the methods of the present invention are used in conjunction with standard accepted clinical methods to diagnose ocular disease. By ocular disease or conditions related to ocular disease is meant a disease, condition of, or injury to the eye. The term ocular disease encompasses glaucoma (e.g., primary open angle glaucoma, normal pressure glaucoma, and pseudoexfoliative glaucoma), and both wet and dry macular degeneration.

The sample is any sample derived from a subject which contains RNA. For example the sample is blood, a blood fraction, body fluid, a population of cells or tissue from the subject. Optionally one or more other samples can be taken over an interval of time that is at least one month between the first sample and the one or more other samples, or taken over an interval of time that is at least twelve months between the first sample and the one or more samples, or they may be taken pre-therapy intervention or post-therapy intervention. In such embodiments, the first sample may be derived from blood and the baseline profile data set may be derived from tissue or body fluid of the subject other than blood. Alternatively, the first sample is derived from tissue or bodily fluid of the subject and the baseline profile data set is derived from blood.

Also included in the invention are kits for the detection of ocular disease in a subject, containing at least one reagent for the detection or quantification of any constituent measured according to the methods of the invention and instructions for using the kit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a graphical representation of the 2-gene model TGFB1 and SERPINB2 based on the Precision Profile™ for Ocular disease (Table 1A), capable of distinguishing between subjects afflicted with normal pressure glaucoma (NPG) and normal subjects, with a discrimination line overlaid onto the graph as an example of the Index Function evaluated at a particular logit value. Values above the line represent subjects predicted to be in the normal population. Values below the line represent subjects predicted to be in the NPG population TGFB1 values are plotted along the Y-axis, SERPINB2 values are plotted along the X-axis.

FIG. 2 is a graphical representation of the 2-gene model MMP19 and CD69, based on the Precision Profile™ for Ocular disease (Table 1A), capable of distinguishing between subjects afflicted with primary open angle glaucoma (POAG) and normal subjects, with a discrimination line overlaid onto the graph as an example of the Index Function evaluated at a particular logit value. Values above the line represent subjects predicted to be in the normal population. Values below the line represent subjects predicted to be in the POAG population. MMP19 values are plotted along the Y-axis, CD69 values are plotted along the X-axis.

FIG. 3 is a graphical representation of the 2-gene model TGFB1 and CD69, based on the Precision Profile™ for Ocular disease (Table 1A), capable of distinguishing between subjects afflicted with normal pressure glaucoma (NPG) and primary open angle glaucoma (POAG) versus normal subjects, with a discrimination line overlaid onto the graph as an example of the Index Function evaluated at a particular logit value. Values above the line represent subjects predicted to be in the normal population. Values below the line represent subjects predicted to be in the NPG and POAG population. TGFB1 values are plotted along the Y-axis, CD69 values are are plotted along the X-axis.

### DETAILED DESCRIPTION

### Definitions

The following terms shall have the meanings indicated unless the context otherwise requires:

“Accuracy” refers to the degree of conformity of a measured or calculated quantity (a test reported value) to its actual (or true) value. Clinical accuracy relates to the proportion of true outcomes (true positives (TP) or true negatives (TN)) versus misclassified outcomes (false positives (FP) or false negatives (FN)), and may be stated as a sensitivity, specificity, positive predictive values (PPV) or negative predictive values (NPV), or as a likelihood, odds ratio, among other measures.

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus.

“Amplification” in the context of a quantitative RT-PCR assay is a function of the number of DNA replications that are required to provide a quantitative determination of its concentration. “Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar.

A “baseline profile data set” is a set of values associated with constituents of a Gene Expression Panel (Precision Profile™) resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “biological condition” of a subject is the condition of the subject in a pertinent realm that is under observation, and such realm may include any aspect of the subject capable of being monitored for change in condition, such as health; disease including ocular disease; cancer; trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Body fluid” of a subject includes blood, urine, spinal fluid, lymph, mucosal secretions, prostatic fluid, semen, haemolymph or any other body fluid known in the art for a subject.

“Calibrated profile data set” is a function of a member of a first profile data set and a corresponding member of a baseline profile data set for a given constituent in a panel.

A “clinical indicator” is any physiological datum used alone or in conjunction with other data in evaluating the physiological condition of a collection of cells or of an organism. This term includes pre-clinical indicators.

“Clinical parameters” encompasses all non-sample or non-Precision Profiles™ of a subject's health status or other characteristics, such as, without limitation, age (AGE), ethnicity (RACE), gender (SEX), and family history of ocular disease.

A “composition” includes a chemical compound, a nutraceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

To “derive” a profile data set from a sample includes determining a set of values associated with constituents of a Gene Expression Panel (Precision Profile™) either (i) by direct measurement of such constituents in a biological sample. “Distinct RNA or protein constituent” in a panel of constituents is a distinct expressed product of a gene, whether RNA or protein. An “expression” product of a gene includes the gene product whether RNA or protein resulting from translation of the messenger RNA.

“FN” is false negative, which for a disease state test means classifying a disease subject incorrectly as non-disease or normal.

“FP” is false positive, which for a disease state test means classifying a normal subject incorrectly as having disease.

A “formula,” “algorithm,” or “model” is any mathematical equation, algorithmic, analytical or programmed process, statistical technique, or comparison, that takes one or more continuous or categorical inputs (herein called “parameters”) and calculates an output value, sometimes referred to as an “index” or “index value.” Non-limiting examples of “formulas” include comparisons to reference values or profiles, sums, ratios, and regression operators, such as coefficients or exponents, value transformations and normalizations (including, without limitation, those normalization schemes based on clinical parameters, such as gender, age, or ethnicity), rules and guidelines, statistical classification models, and neural networks trained on historical populations. Of particular use in combining constituents of a Gene Expression Panel (Precision Profile™) are linear and non-linear equations and statistical significance and classification analyses to determine the relationship between levels of constituents of a Gene Expression Panel (Precision Profile™) detected in a subject sample and the subject's risk of ocular disease. In panel and combination construction, of particular interest are structural and synactic statistical classification algorithms, and methods of risk index construction, utilizing pattern recognition features, including, without limitation, such established techniques such as cross-correlation, Principal Components Analysis (PCA), factor rotation, Logistic Regression Analysis (LogReg), Kolmogorov Smirnoff tests (KS), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques (CART, LART, LARTree, FlexTree, amongst others), Shrunken Centroids (SC), StepAIC, K-means, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art. Many of these techniques are useful either combined with a consituentes of a Gene Expression Panel (Precision Profile™) selection technique, such as forward selection, backwards selection, or stepwise selection, complete enumeration of all potential panels of a given size, genetic algorithms, voting and committee methods, or they may themselves include biomarker selection methodologies in their own technique. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other clinical studies, or cross-validated within the study they were originally trained in, using such techniques as Bootstrap, Leave-One-Out (LOO) and 10-Fold cross-validation (10-Fold CV). At various steps, false discovery rates (FDR) may be estimated by value permutation according to techniques known in the art.

A “Gene Expression Panel” (Precision Profile™) is an experimentally verified set of constituents, each constituent being a distinct expressed product of a gene, whether RNA or protein, wherein constituents of the set are selected so that their measurement provides a measurement of a targeted biological condition.

A “Gene Expression Profile” (Precision Profile™) is a set of values associated with constituents of a Gene Expression Panel resulting from evaluation of a biological sample (or population or set of samples).

A “Gene Expression Profile Inflammation Index” is the value of an index function that provides a mapping from an instance of a Gene Expression Profile into a single-valued measure of inflammatory condition.

A Gene Expression Profile Ocular Disease Index” is the value of an index function that provides a mapping from an instance of a Gene Expression Profile into a single-valued measure of an ocular disease condition.

The “health” of a subject includes mental, emotional, physical, spiritual, allopathic, naturopathic and homeopathic condition of the subject.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response initiated or sustained by any number of chemical, physical or biological agents or combination of agents.

“Inflammatory state” is used to indicate the relative biological condition of a subject resulting from inflammation, or characterizing the degree of inflammation.

A “large number” of data sets based on a common panel of genes is a number of data sets sufficiently large to permit a statistically significant conclusion to be drawn with respect to an instance of a data set based on the same panel.

“Negative predictive value” or “NPV” is calculated by TN/(TN+FN) or the true negative fraction of all negative test results. It also is inherently impacted by the prevalence of the disease and pre-test probability of the population intended to be tested.

See, e.g., O'Marcaigh A S, Jacobson R M, “Estimating the Predictive Value of a Diagnostic Test, How to Prevent Misleading or Confusing Results,” Clin. Ped. 1993, 32(8): 485-491, which discusses specificity, sensitivity, and positive and negative predictive values of a test, e.g., a clinical diagnostic test. Often, for binary disease state classification approaches using a continuous diagnostic test measurement, the sensitivity and specificity is summarized by Receiver Operating Characteristics (ROC) curves according to Pepe et al., “Limitations of the Odds Ratio in Gauging the Performance of a Diagnostic, Prognostic, or Screening Marker,” Am. J. Epidemiol 2004, 159 (9): 882-890, and summarized by the Area Under the Curve (AUC) or c-statistic, an indicator that allows representation of the sensitivity and specificity of a test, assay, or method over the entire range of test (or assay) cut points with just a single value. See also, e.g., Shultz, “Clinical Interpretation of Laboratory Procedures,” chapter 14 in Teitz, Fundamentals of Clinical Chemistry, Burns and Ashwood (eds.), 4th edition 1996, W.B. Saunders Company, pages 192-199; and Zweig et al., “ROC Curve Analysis: An Example Showing the Relationships Among Serum Lipid and Apolipoprotein Concentrations in Identifying Subjects with Coronory Artery Disease,” Clin. Chem., 1992, 38(8): 1425-1428. An alternative approach using likelihood functions, BIC, odds ratios, information theory, predictive values, calibration (including goodness-of-fit), and reclassification measurements is summarized according to Cook, “Use and Misuse of the Receiver Operating Characteristic Curve in Risk Prediction,” Circulation 2007, 115: 928-935.

A “normal” subject is a subject who is generally in good health, has not been diagnosed with ocular disease, or one who is not suffering from ocular disease, is asymptomatic for ocular disease, and lacks the traditional laboratory risk factors for ocular disease.

A “normative” condition of a subject to whom a composition is to be administered means the condition of a subject before administration, even if the subject happens to be suffering from a disease.

The term “ocular disease” is used to indicate a disease or condition of, or injury to, the eye. As defined herein, ocular disease encompasses glaucoma (e.g., primary open angle glaucoma, normal pressure glaucoma, pseudoexfoliative glaucoma, primary angle closure glaucoma, and pigmentary glaucoma), age-related macular degeneration (wet and dry), retinal detachment, retinoschisis, retinopathy (prematurity, hypertensive, diabetic, and proliferative vitreo-retinopathy), retinitis pigmentosa, macular edema, scleritis, keratitis, corneal ulcer, Fuch's dystrophy, iritis, keratoconus, keratoconjunctivitis sicca, uveitis, conjunctivitis, and cataract.

A “panel” of genes is a set of genes including at least two constituents.

A “population of cells” refers to any group of cells wherein there is an underlying commonality or relationship between the members in the population of cells, including a group of cells taken from an organism or from a culture of cells or from a biopsy, for example.

“Positive predictive value” or “PPV” is calculated by TP/(TP+FP) or the true positive fraction of all positive test results. It is inherently impacted by the prevalence of the disease and pre-test probability of the population intended to be tested.

“Risk” in the context of the present invention, relates to the probability that an event will occur over a specific time period, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of lower risk cohorts, across population divisions (such as tertiles, quartiles, quintiles, or deciles, etc.) or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion.

“Risk evaluation,” or “evaluation of risk” in the context of the present invention encompasses making a prediction of the probability, odds, or likelihood that an event or disease state may occur, and/or the rate of occurrence of the event or conversion from one disease state to another, i.e., from a normal condition to ocular disease and vice versa. Risk evaluation can also comprise prediction of future clinical parameters, traditional laboratory risk factor values, or other indices of ocular disease results, either in absolute or relative terms in reference to a previously measured population. Such differing use may require different consituentes of a Gene Expression Panel (Precision Profile™) combinations and individualized panels, mathematical algorithms, and/or cut-off points, but be subject to the same aforementioned measurements of accuracy and performance for the respective intended use.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art. The sample is blood, urine, spinal fluid, lymph, mucosal secretions, prostatic fluid, semen, haemolymph or any other body fluid known in the art for a subject. The sample is also a tissue sample.

“Sensitivity” is calculated by TP/(TP+FN) or the true positive fraction of disease subjects.

“Specificity” is calculated by TN/(TN+FP) or the true negative fraction of non-disease or normal subjects.

By “statistically significant”, it is meant that the alteration is greater than what might be expected to happen by chance alone (which could be a “false positive”). Statistical significance can be determined by any method known in the art. Commonly used measures of significance include the p-value, which presents the probability of obtaining a result at least as extreme as a given data point, assuming the data point was the result of chance alone. A result is often considered highly significant at a p-value of 0.05 or less and statistically significant at a p-value of 0.10 or less. Such p-values depend significantly on the power of the study performed.

A “set” or “population” of samples or subjects refers to a defined or selected group of samples or subjects wherein there is an underlying commonality or relationship between the members included in the set or population of samples or subjects.

A “Signature Profile” is an experimentally verified subset of a Gene Expression Profile selected to discriminate a biological condition, agent or physiological mechanism of action.

A “Signature Panel” is a subset of a Gene Expression Panel (Precision Profile™), the constituents of which are selected to permit discrimination of a biological condition, agent or physiological mechanism of action.

A “subject” is a cell, tissue, or organism, human or non-human, whether in vivo, ex vivo or in vitro, under observation. As used herein, reference to evaluating the biological condition of a subject based on a sample from the subject, includes using blood or other tissue sample from a human subject to evaluate the human subject's condition; it also includes, for example, using a blood sample itself as the subject to evaluate, for example, the effect of therapy or an agent upon the sample.

A “stimulus” includes (i) a monitored physical interaction with a subject, for example ultraviolet A or B, or light therapy for seasonal affective disorder, or treatment of psoriasis with psoralen or treatment of cancer with embedded radioactive seeds, other radiation exposure, and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

“TN” is true negative, which for a disease state test means classifying a non-disease or normal subject correctly.

“TP” is true positive, which for a disease state test means correctly classifying a disease subject.

The PCT patent application publication number WO 01/25473, published Apr. 12, 2001, entitled “Systems and Methods for Characterizing a Biological Condition or Agent Using Calibrated Gene Expression Profiles,” which is herein incorporated by reference, discloses the use of Gene Expression Panels (Precision Profiles™) for the evaluation of (i) biological condition (including with respect to health and disease) and (ii) the effect of one or more agents on biological condition (including with respect to health, toxicity, therapeutic treatment and drug interaction).

In particular, the Gene Expression Panels (Precision Profiles™) described herein may be used, without limitation, for measurement of the following: therapeutic efficacy of natural or synthetic compositions or stimuli that may be formulated individually or in combinations or mixtures for a range of targeted biological conditions; prediction of toxicological effects and dose effectiveness of a composition or mixture of compositions for an individual or for a population or set of individuals or for a population of cells; determination of how two or more different agents administered in a single treatment might interact so as to detect any of synergistic, additive, negative, neutral or toxic activity; performing pre-clinical and clinical trials by providing new criteria for pre-selecting subjects according to informative profile data sets for revealing disease status; and conducting preliminary dosage studies for these patients prior to conducting phase 1 or 2 trials. These Gene Expression Panels (Precision Profiles™) may be employed with respect to samples derived from subjects in order to evaluate their biological condition.

The present invention provides Gene Expression Panels (Precision Profiles™) for the evaluation or characterization of ocular disease and conditions related to ocular disease in a subject. In addition, the Gene Expression Panels described herein also provide for the evaluation of the effect of one or more agents for the treatment of ocular disease and conditions related to ocular disease.

The Gene Expression Panels (Precision Profiles™) are referred to herein as the “Precision

Profile™ for Ocular Disease” and the “Precision Profile™ for Inflammatory Response”. A Precision Profile™ for Ocular Disease includes one or more genes, e.g., constituents, listed in Tables 1, 3-5, 7-9, and 11-13, whose expression is associated with ocular disease or conditions related to ocular disease. A Precision Profile™ for Inflammatory Response includes one or more genes, e.g., constituents, listed in Table 2, whose expression is associated with inflammatory response and ocular disease. Each gene of the Precision Profile™ for Ocular Disease and Precision Profile™ for Inflammatory Response is referred to herein as an ocular disease associated gene or an ocular disease associated constituent.

It has been discovered that valuable and unexpected results may be achieved when the quantitative measurement of constituents is performed under repeatable conditions (within a degree of repeatability of measurement of better than twenty percent, preferably ten percent or better, more preferably five percent or better, and more preferably three percent or better). For the purposes of this description and the following claims, a degree of repeatability of measurement of better than twenty percent may be used as providing measurement conditions that are “substantially repeatable”. In particular, it is desirable that each time a measurement is obtained corresponding to the level of expression of a constituent in a particular sample, substantially the same measurement should result for substantially the same level of expression. In this manner, expression levels for a constituent in a Gene Expression Panel (Precision Profile™) may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar as defined herein. When both of these criteria are satisfied, then measurement of the expression level of one constituent may be meaningfully compared with measurement of the expression level of another constituent in a given sample and from sample to sample.

The evaluation or characterization of ocular disease is defined to be diagnosing ocular disease, assessing the presence or absence of ocular disease, assessing the risk of developing ocular disease, or assessing the prognosis of a subject with ocular disease. Similarly, the evaluation or characterization of an agent for treatment of ocular disease includes identifying agents suitable for the treatment of ocular disease. The agents can be compounds known to treat ocular disease or compounds that have not been shown to treat ocular disease.

Ocular disease and conditions related to ocular disease is evaluated by determining the level of expression (e.g., a quantitative measure) of an effective number (e.g., one or more) of constituents of a Gene Expression Panel (Precision Profile™) disclosed herein (i.e., Tables 1-2). By an effective number is meant the number of constituents that need to be measured in order to discriminate between a normal subject and a subject having ocular disease. Preferably the constituents are selected as to discriminate between a normal subject and a subject having ocular disease with at least 75% accuracy, more preferably 80%, 85%, 90%, 95%, 97%, 98%, 99% or greater accuracy.

The level of expression is determined by any means known in the art, such as for example quantitative PCR. The measurement is obtained under conditions that are substantially repeatable. Optionally, the qualitative measure of the constituent is compared to a reference or baseline level or value (e.g. a baseline profile set). In one embodiment, the reference or baseline level is a level of expression of one or more constituents in one or more subjects known not to be suffering from ocular disease (e.g., normal, healthy individual(s)). Alternatively, the reference or baseline level is derived from the level of expression of one or more constituents in one or more subjects known to be suffering from ocular disease. Optionally, the baseline level is derived from the same subject from which the first measure is derived. For example, the baseline is taken from a subject prior to receiving treatment or surgery for ocular disease, or at different time periods during a course of treatment. Such methods allow for the evaluation of a particular treatment for a selected individual. Comparison can be performed on test (e.g., patient) and reference samples (e.g., baseline) measured concurrently or at temporally distinct times. An example of the latter is the use of compiled expression information, e.g., a gene expression database, which assembles information about expression levels of ocular disease associated genes.

A reference or baseline level or value as used herein can be used interchangeably and is meant to be relative to a number or value derived from population studes, including without limitation, such subjects having similar age range, subjects in the same or similar ethnic group, sex, or, in female subjects, pre-menopausal or post-menopausal subjects, or relative to the starting sample of a subject undergoing treatment for ocular disease. Such reference values can be derived from statistical analyses and/or risk prediction data of populations obtained from mathematical algorithms and computed indices of ocular disease. Reference indices can also be constructed and used using algorithms and other methods of statistical and structural classification.

In one embodiment of the present invention, the reference or baseline value is the amount of expression of an ocular disease associated gene in a control sample derived from one or more subjects who are both asymptomatic and lack traditional laboratory risk factors for ocular disease.

In another embodiment of the present invention, the reference or baseline value is the level of ocular disease associated genes in a control sample derived from one or more subjects who are not at risk or at low risk for developing ocular disease.

In a further embodiment, such subjects are monitored and/or periodically retested for a diagnostically relevant period of time (“longitudinal studies”) following such test to verify continued absence from ocular disease. Such period of time may be one year, two years, two to five years, five years, five to ten years, ten years, or ten or more years from the initial testing date for determination of the reference or baseline value. Furthermore, retrospective measurement of ocular disease associated genes in properly banked historical subject samples may be used in establishing these reference or baseline values, thus shortening the study time required, presuming the subjects have been appropriately followed during the intervening period through the intended horizon of the product claim.

A reference or baseline value can also comprise the amounts of ocular disease associated genes derived from subjects who show an improvement in ocular disease status as a result of treatments and/or therapies for the ocular disease being treated and/or evaluated.

In another embodiment, the reference or baseline value is an index value or a baseline value. An index value or baseline value is a composite sample of an effective amount of ocular disease associated genes from one or more subjects who do not have ocular disease.

For example, where the reference or baseline level is comprised of the amounts of ocular disease associated genes derived from one or more subjects who have not been diagnosed with ocular disease or are not known to be suffering from ocular disease, a change (e.g., increase or decrease) in the expression level of a ocular disease associated gene in the patient-derived sample of an ocular disease associated gene compared to the expression level of such gene in the reference or baseline level indicates that the subject is suffering from or is at risk of developing ocular disease. In contrast, when the methods are applied prophylacticly, a similar level of expression in the patient-derived sample of an ocular disease associated gene as compared to such gene in the baseline level indicates that the subject is not suffering from or at risk of developing ocular disease.

Where the reference or baseline level is comprised of the amounts of ocular disease associated genes derived from one or more subjects who have been diagnosed with ocular disease, or are known to be suffering from ocular disease, a similarity in the expression pattern in the patient-derived sample of an ocular disease associated gene compared to the ocular disease baseline level indicates that the subject is suffering from or is at risk of developing ocular disease.

Expression of an ocular disease associated gene also allows for the course of treatment of ocular disease to be monitored. In this method, a biological sample is provided from a subject undergoing treatment, e.g., if desired, biological samples are obtained from the subject at various time points before, during, or after treatment. Expression of an ocular disease associated gene is then determined and compared to a reference or baseline profile. The baseline profile may be taken or derived from one or more individuals who have been exposed to the treatment. Alternatively, the baseline level may be taken or derived from one or more individuals who have not been exposed to the treatment. For example, samples may be collected from subjects who have received initial treatment for ocular disease and subsequent treatment for ocular disease to monitor the progress of the treatment.

Differences in the genetic makeup of individuals can result in differences in their relative abilities to metabolize various drugs. Accordingly, the Precision Profile™ for Ocular Disease (Table 1A and 1B) and the Precision Profile' for Inflammatory Response (Table 2) disclosed herein allow for a putative therapeutic or prophylactic to be tested from a selected subject in order to determine if the agent is a suitable for treating or preventing ocular disease in the subject. Additionally, other genes known to be associated with toxicity may be used. By suitable for treatment is meant determining whether the agent will be efficacious, not efficacious, or toxic for a particular individual. By toxic it is meant that the manifestations of one or more adverse effects of a drug when administered therapeutically. For example, a drug is toxic when it disrupts one or more normal physiological pathways.

To identify a therapeutic that is appropriate for a specific subject, a test sample from the subject is exposed to a candidate therapeutic agent, and the expression of one or more of ocular disease genes is determined. A subject sample is incubated in the presence of a candidate agent and the pattern of ocular disease associated gene expression in the test sample is measured and compared to a baseline profile, e.g., an ocular disease baseline profile or a non-ocular disease baseline profile or an index value. The test agent can be any compound or composition. For example, the test agent is a compound known to be useful in the treatment of ocular disease. Alternatively, the test agent is a compound that has not previously been used to treat ocular disease.

If the reference sample, e.g., baseline is from a subject that does not have ocular disease a similarity in the pattern of expression of ocular disease genes in the test sample compared to the reference sample indicates that the treatment is efficacious. Whereas a change in the pattern of expression of ocular disease genes in the test sample compared to the reference sample indicates a less favorable clinical outcome or prognosis. By “efficacious” is meant that the treatment leads to a decrease of a sign or symptom of ocular disease in the subject or a change in the pattern of expression of an ocular disease associated gene such that the gene expression pattern has an increase in similarity to that of a reference or baseline pattern. Assessment of ocular disease is made using standard clinical protocols. Efficacy is determined in association with any known method for diagnosing or treating ocular disease.

A Gene Expression Panel (Precision Profile™) is selected in a manner so that quantitative measurement of RNA or protein constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel (Precision Profile™) and (ii) a baseline quantity.

Additional embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; (d) to monitor a biological condition of a subject; (e) for measurement of therapeutic efficacy of natural or synthetic compositions or stimuli that may be formulated individually or in combinations or mixtures for a range of targeted biological conditions; (f) for predictions of toxicological effects and dose effectiveness of a composition or mixture of compositions for an individual or for a population or set of individuals or for a population of cells; (g) for determination of how two or more different agents administered in a single treatment might interact so as to detect any of synergistic, additive, negative, neutral of toxic activity (h) for performing pre-clinical and clinical trials by providing new criteria for pre-selecting subjects according to informative profile data sets for revealing disease status and conducting preliminary dosage studies for these patients prior to conducting Phase 1 or 2 trials.

Gene expression profiling and the use of index characterization for a particular condition or agent or both may be used to reduce the cost of Phase 3 clinical trials and may be used beyond Phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; managing the health care of a patient; and quality control for different batches of an agent or a mixture of agents.

### The Subject

The methods disclosed herein may be applied to cells of humans, mammals or other organisms without the need for undue experimentation by one of ordinary skill in the art because all cells transcribe RNA and it is known in the art how to extract RNA from all types of cells.

A subject can include those who have not been previously diagnosed as having ocular disease or a condition related to ocular disease. Alternatively, a subject can also include those who have already been diagnosed as having ocular disease or a condition related to ocular disease. Diagnosis of an ocular disease such as glaucoma is made, for example, from any one or combination of the following procedures: 1) measurement of intraolcular pressure; 2) examination of the appearance of the meshwork; 3) examination of the appearance of the optic nerve; 4) examination of the individual's visual field, particularly peripheral vision. Diagnosis of an ocular disease such as AMD is made, for example, from any one or combination of the following procedures: a retinal examination, a visual test using an Amsler grid which detects changes in central vision (a sign of AMD if the grid appears distorted); and fluorescein angiography to specifically examine the retinal blood vessels surrounding the macula.

Optionally, the subject has previously been treated with a therapeutic agent, including but not limited to therapeutic agents for the treatment of glaucoma, such as beta blockers (e.g., Timoptic, Betoptic), topical beta-adrenergic receptor antagonists (e.g., timolol, levobunolol (Betagan), and betaxolol), carbonic anhydrase inhibitors (e.g., dorzolamide (Trusopt), brinzolamide (Azopt), and acetazolamide (Diamox)), alpha2-adrenergic agonists (e.g., brimonidine (Alphagan)); prostaglandin (e.g., latanoprost (Xalatan), bimatoprost (Lumigan) and travoprost (Travatan)), sympathomimetics (e.g., epinephrine and dipivefrin (Propine)), miotic agents (parasympathomimetics, e.g., pilocarpine), and marijuana; and therapeutic agents for the treatment of wet AMD, such as pegabtanib (Macugen), verteporfin (Visudyne), bevacizumab (Avastin), ranibizumab (Lucentis), anecortave (Retaane), squalamine (Evizon), siRNA, and antisense oligonucleotides iCo-007 (targeting the Raf-1 kinase). Optionally, the therapeutic agent is administered alone, or in combination, or in succession with a surgical procedure for treating ocular disease, including but not limited to laser surgery, photodynamic therapy, open, incisional surgery, radiation therapy (brachytherapy) and rheopheresis. For example, an argon laser may be used to perform a procedure called a trabeculoplasty, where the laser is focused into the meshwork where it alters cells there to let aqueous fluid leave the eye more efficiently. A laser may also be used to make a small hole in the colored part of the eye (the iris) to allow the aqueous fluid to flow more freely within in the eye. A laser or freezing treatment may also be used to destroy tissue in the eye that makes aqueous humor. Open, incisional surgery may be performed if medication and initial laser treatments are unsuccessful in reducing pressure within the eye. One type of surgery, a trabeculectomy, creates an opening in the wall of the eye so that aqueous humor can drain. Another type of surgery places a drainage tube into the eye between the cornea and iris. It exits at the junction of the cornea and sclera (the white portion of the eye). The tube drains to a plate that is sewn on the surface of the eye about halfway back.

A subject can also include those who are suffering from, or at risk of developing ocular disease or a condition related to ocular disease, such as those who exhibit known risk factors for ocular disease or conditions related to ocular disease. For example, known risk factors for ocular disease such as glaucoma include but are not limited to: heredity, race (high prevalence among African Americans), suspicious optic nerve appearance (cupping >50% or assymetry), central corneal thickness less than 555 microns (0.5 mm), gender (increased risk in males), aging (being older than 60), diabetes, high mypoia (nearsightedness), high blood pressure (hypertension), frequent migraines, an injury or surgery to the eye, and a history of steroid use. Known risk factors for developing AMD include aging, smoking, gender (women appear to be at slightly higher risk), obesity, hypertension, lighter eye color, heredity, and race. There are also suggestions that visible and ultraviolet light may damage the retina, and that low consumption of fruits and vegetables, which contain certain antioxidants may potentially increase risk of AMD.

Selecting Constituents of a Gene Expression Panel (Precision Profile™)

The general approach to selecting constituents of a Gene Expression Panel (Precision Profile™) has been described in PCT application publication number WO 01/25473, incorporated herein by reference in its entirety. A wide range of Gene Expression Panels (Precision Profiles™) have been designed and experimentally validated, each panel providing a quantitative measure of biological condition that is derived from a sample of blood or other tissue. For each panel, experiments have verified that a Gene Expression Profile using the panel's constituents is informative of a biological condition. (It has also been demonstrated that in being informative of biological condition, the Gene Expression Profile is used, among other things, to measure the effectiveness of therapy, as well as to provide a target for therapeutic intervention.).

Tables 1-5, 7-9, and 11-13 listed below, include relevant genes which may be selected for a given Precision Profile™, such as the Precision Profiles™ demonstrated herein to be useful in the evaluation of ocular disease and conditions related to ocular disease. Tables 1A and 1B are panels of 96 and 97 genes respectively, whose expression is associated with ocular disease or conditions related to ocular disease.

Table 2 is a panel of genes whose expression is associated with inflammatory response. Inflammation is known to play a critical role in many types of ocular diseases. The earliest events of inflammation are related to hyperemia and effusion of fluid from blood vessels responding to locally-generated inflammatory mediators. In most tissues such serous effusion is of little consequence, but the anatomy of the eye presents some special problems. Serous effusion from the choroid, for example, creates instantly blinding retinal detachment that might ultimately result in irreversible retinal damage because the retina is separated from its nutritional choroidal support. Alternatively, the leakage of protein into the aqueous humor changes its optical properties and results in aqueous flare, and the abnormal chemical composition of the aqueous is a potential cause for cataract because the lens depends entirely upon the delivery of quantitatively and qualitatively normal aqueous humor for its nutritional health.

In some instances, the leakage of small molecular weight proteins from reactive vessels is followed by the leakage of larger proteins like fibrinogen, resulting in the extravascular accumulation of fibrin. The potential for adhesion between adjacent inflamed, sticky surfaces is little more than an inconvenience in most tissues, but within the globe the adhesion of iris to lens creates posterior synechia with the potential for pupillary block, iris bombe, and secondary glaucoma. Similarly, the accumulation and subsequent contraction of fibrin within the vitreous creates the risk of traction retinal detachment.

Additionally, leukocytes may accumulate and settle by gravity within the anterior chamber as they attempt to exit the globe via the trabecular meshwork (hypopyon), or form adherent clusters that stick to the corneal endothelium (keratic precipitates). Because the globe is a closed sphere, inflammatory mediators and various cytokines associated with leucocytic recruitment or subsequent events of wound healing are distributed throughout the globe, so there is really no such thing as localized intraocular inflammation. Although, for example, the anterior uveitis is clinically distinguishable from choroiditis, from a histologic perspective all intraocular inflammation is diffuse (i.e. endophthalmitis). As such, both the ocular disease genes listed in Tables 1A and 1B and the inflammatory response genes listed in Table 2 can be used to detect ocular disease and distinguish between subjects suffering from ocular disease and normal subjects.

Table 5 was derived from a study of the gene expression patterns described in Example 1 below. Table 5 describes a multi-gene model based on genes from the Precision Profile™ for Ocular Disease (Glaucoma) (shown in Table 1A), derived from latent class modeling of the subjects from this study using 1 and 2 gene models to distinguish between subjects suffering from normal pressure glaucoma (NPG) and normal subjects. Constituent models selected from Table 5 are capable of correctly classifying ocular disease-afflicted and/or normal subjects with at least 75% accuracy. For example, in Table 5, Gene Column 1, it can be seen that the 1-gene model, TGFB1, correctly classifies NPG-afflicted subjects with 100% accuracy, and normal subjects with 92% accuracy. In Table 5, Gene Column 2, it can be seen that the 2-gene model, TGFB1 and SERPINB2, correctly classifies NPG-afflicted subjects with 100% accuracy, and normal subjects with 92% accuracy.

Table 9 was derived from a study of the gene expression patterns described in Example 2 below. Table 9 also describes multi-gene models based on genes from the Precision Profile™ for Ocular Disease (Glaucoma) (shown in Table 1A), derived from latent class modeling of the subjects from this study using 1 and 2-gene models to distinguish between subjects suffering from primary open angle glaucoma (POAG) based on genes from the Precision Profile™ for Ocular Disease (Table 1A). Constituent models selected from Table 9 are capable of correctly classifying POAG-afflicted and/or normal subjects with at least 75% accuracy. For example, in Table 9, Gene Column 1, it can be seen that the 1-gene model, MMP19, correctly classifies POAG-afflicted subjects with 82% accuracy, and normal subjects with 83% accuracy. In Table 9, Gene Column 2, it can be seen that the 2-gene model, MMP19 and CD69, correctly classifies POAG-afflicted subjects with 94% accuracy, and normal subjects with 92% accuracy.

Table 13 was derived from a study of the gene expression patterns described in Example 3 below. Table 13 also describes multi-gene models based on genes from the Precision Profile™ for Ocular Disease (Glaucoma) (shown in Table 1A), derived from latent class modeling of the subjects from this study using 1 and 2-gene models to distinguish between subjects suffering from both normal pressure glaucoma (NPG) and primary open angle glaucoma (POAG) based on genes from the Precision Profile™ for Ocular Disease (Table 1A). Constituent models selected from Table 13 are capable of correctly classifying NPG and POAG-afflicted and/or normal subjects with at least 75% accuracy. For example, in Table 13, Gene Column 1, it can be seen that the 1-gene model, TGFB1, correctly classifies NPG and POAG-afflicted subjects with 85% accuracy, and normal subjects with 92% accuracy. In Table 13, Gene Column 2, it can be seen that the 2-gene model, TGFB1 and CD69, correctly classifies NPG and POAG-afflicted subjects with 94% accuracy, and normal subjects with 92% accuracy.

In general, panels may be constructed and experimentally validated by one of ordinary skill in the art in accordance with the principles articulated in the present application.

### Design of Assays

Typically, a sample is run through a panel in replicates of three for each target gene (assay); that is, a sample is divided into aliquots and for each aliquot the concentrations of each constituent in a Gene Expression Panel (Precision Profile™) is measured. From over thousands of constituent assays, with each assay conducted in triplicate, an average coefficient of variation was found (standard deviation/average)*100, of less than 2 percent among the normalized ΔC_{T} measurements for each assay (where normalized quantitation of the target mRNA is determined by the difference in threshold cycles between the internal control (e.g., an endogenous marker such as 18S rRNA, or an exogenous marker) and the gene of interest. This is a measure called “intra-assay variability”. Assays have also been conducted on different occasions using the same sample material. This is a measure of “inter-assay variability”. Preferably, the average coefficient of variation of intra-assay variability or inter-assay variability is less than 20%, more preferably less than 10%, more preferably less than 5%, more preferably less than 4%, more preferably less than 3%, more preferably less than 2%, and even more preferably less than 1%.

It has been determined that it is valuable to use the quadruplicate or triplicate test results to identify and eliminate data points that are statistical “outliers”; such data points are those that differ by a percentage greater, for example, than 3% of the average of all three or four values. Moreover, if more than one data point in a set of three or four is excluded by this procedure, then all data for the relevant constituent is discarded.

### Measurement of Gene Expression for a Constituent in the Panel

For measuring the amount of a particular RNA in a sample, methods known to one of ordinary skill in the art were used to extract and quantify transcribed RNA from a sample with respect to a constituent of a Gene Expression Panel (Precision Profile™). (See detailed protocols below. Also see PCT application publication number WO 98/24935 herein incorporated by reference for RNA analysis protocols). Briefly, RNA is extracted from a sample such as any tissue, body fluid, cell, or culture medium in which a population of cells of a subject might be growing. For example, cells may be lysed and RNA eluted in a suitable solution in which to conduct a DNAse reaction. Subsequent to RNA extraction, first strand synthesis may be performed using a reverse transcriptase. Gene amplification, more specifically quantitative PCR assays, can then be conducted and the gene of interest calibrated against an internal marker such as 18S rRNA (Hirayama et al., Blood 92, 1998: 46-52). Any other endogenous marker can be used, such as 28S-25S rRNA and 5S rRNA. Samples are measured in multiple replicates, for example, 3 replicates. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, quantitation of the reporter signal for an internal marker generated by the exponential increase of amplified product may also be used. Amplification of the target template may be accomplished by isothermic gene amplification strategies or by gene amplification by thermal cycling such as PCR.

It is desirable to obtain a definable and reproducible correlation between the amplified target or reporter signal, i.e., internal marker, and the concentration of starting templates. It has been discovered that this objective can be achieved by careful attention to, for example, consistent primer-template ratios and a strict adherence to a narrow permissible level of experimental amplification efficiencies (for example 80.0 to 100%+/−5% relative efficiency, typically 90.0 to 100%+/−5% relative efficiency, more typically 95.0 to 100%+/−2%, and most typically 98 to 100%+/−1% relative efficiency). In determining gene expression levels with regard to a single Gene Expression Profile, it is necessary that all constituents of the panels, including endogenous controls, maintain similar amplification efficiencies, as defined herein, to permit accurate and precise relative measurements for each constituent. Amplification efficiencies are regarded as being “substantially similar”, for the purposes of this description and the following claims, if they differ by no more than approximately 10%, preferably by less than approximately 5%, more preferably by less than approximately 3%, and more preferably by less than approximately 1%. Measurement conditions are regarded as being “substantially repeatable, for the purposes of this description and the following claims, if they differ by no more than approximately +/−10% coefficient of variation (CV), preferably by less than approximately +/−5% CV, more preferably +/−2% CV. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, tests are run to assure that these conditions are satisfied. For example, the design of all primer-probe sets are done in house, experimentation is performed to determine which set gives the best performance. Even though primer-probe design can be enhanced using computer techniques known in the art, and notwithstanding common practice, it has been found that experimental validation is still useful. Moreover, in the course of experimental validation, the selected primer-probe combination is associated with a set of features:

The reverse primer should be complementary to the coding DNA strand. In one embodiment, the primer should be located across an intron-exon junction, with not more than four bases of the three-prime end of the reverse primer complementary to the proximal exon. (If more than four bases are complementary, then it would tend to competitively amplify genomic DNA.)

In an embodiment of the invention, the primer probe set should amplify cDNA of less than 110 bases in length and should not amplify, or generate fluorescent signal from, genomic DNA or transcripts or cDNA from related but biologically irrelevant loci.

A suitable target of the selected primer probe is first strand cDNA, which in one embodiment may be prepared from whole blood as follows:

(a) Use of Cell Systems or Whole Blood for Ex Vivo Assessment of a Biological Condition.

Human blood is obtained by venipuncture and prepared for assay. The aliquots of heparinized, whole blood are mixed with additional test therapeutic compounds and held at 37° C. in an atmosphere of 5% CO₂ for 30 minutes. Cells are lysed and nucleic acids, e.g., RNA, are extracted by various standard means.

Nucleic acids, RNA and/or DNA are purified from cells, tissues or fluids of the test population of cells. Cells systems that may be used to study ocular disease includes trabecular meshwork (typically stimulated with TGFB2), retinal Ganglion cells (induction of apoptosis via neurotrophin deprivation and/or glutamate toxicity; induction of oxidative stress via EGCG, epigallocatechin gallate), optic nerve head cells and choroid epithelial cells (laser induction of neovascularization). RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), in the present using a filter-based RNA isolation system from Ambion (RNAqueous™, Phenol-free Total RNA Isolation Kit, Catalog #1912, version 9908; Austin, Tex.).

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples (see, for example, RT PCR, Chapter 15 in RNA Methodologies, A Laboratory Guide for Isolation and Characterization, 2nd edition, 1998, Robert E. Farrell, Jr., Ed., Academic Press; or Chapter 22 pp. 143-151, RNA Isolation and Characterization Protocols, Methods in Molecular Biology, Volume 86, 1998, R. Rapley and D. L. Manning Eds., Human Press, or 14 in Statistical refinement of primer design parameters, Chapter 5, pp. 55-72, PCR Applications: Protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7900 obtained from Applied Biosystems, Foster City, Calif.; see Nucleic acid detection methods, pp. 1-24, in Molecular Methods for Virus Detection, D. L. Wiedbrauk and D. H., Farkas, Eds., 1995, Academic Press). Amplified nucleic acids are detected using fluorescent-tagged detection oligonucleotide probes (see, for example, Taqman™ PCR Reagent Kit, Protocol, part number 402823, Revision A, 1996, Applied Biosystems, Foster City Calif.) that are identified and synthesized from publicly known databases as described for the amplification primers.

For example without limitation, amplified cDNA is detected and quantified using detection systems such as the ABI Prism® 7900 Sequence Detection System (Applied Biosystems (Foster City, Calif.)), the Cepheid SmartCycler® and Cepheid GeneXpert® Systems, the Fluidigm BioMark™ System, and the Roche LightCycler® 480 Real-Time PCR System. Amounts of specific RNAs contained in the test sample can be related to the relative quantity of fluorescence observed (see for example, Advances in Quantitative PCR Technology: 5′ nuclease assays, Y. S. Lie and C. J. Petropolus, Current Opinion in Biotechnology, 1998, 9:43-48, or Rapid Thermal Cycling and PCR Kinetics, pp. 211-229, chapter 14 in PCR Applications: protocols for functional genomics, M. A. Innis, D. H. Gelfand and J. J. Sninsky, Eds., 1999, Academic Press).

As a particular implementation of the approach described here in detail is a procedure for synthesis of first strand cDNA for use in PCR. Examples of the procedure used with several of the above-mentioned detection systems are described below. In some embodiments, these procedures can be used for both whole blood RNA and RNA extracted from cultured cells (e.g., trabecular meshwork, retinal Ganglion cells, optic nerve head cells and choroid epithelial cells). Methods herein may also be applied using proteins where sensitive quantitative techniques, such as an Enzyme Linked ImmunoSorbent Assay (ELISA) or mass spectroscopy, are available and well-known in the art for measuring the amount of a protein constituent (see WO 98/24935 herein incorporated by reference).

An example of a procedure of the synthesis of first strand cDNA for use in PCR amplification is as follows:

Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent)

Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| | |
|---|---|
| 1 reaction (mL) | 11X, e.g. 10 samples (μL) |

| | | | |
|---|---|---|---|
| 10X RT Buffer | 10.0 | 110.0 | |
| 25 mM MgCl₂ | 22.0 | 242.0 | |
| dNTPs | 20.0 | 220.0 | |
| Random Hexamers | 5.0 | 55.0 | |
| RNAse Inhibitor | 2.0 | 22.0 | |
| Reverse Transcriptase | 2.5 | 27.5 | |
| Water | 18.5 | 203.5 | |
| Total: | 80.0 | 880.0 | (80 μL per sample) |

4. Bring each RNA sample to a total volume of 20 μL in a 1.5 mL microcentrifuge tube (remove 10 μL RNA and dilute to 20 μL with RNase/DNase free water, for whole blood RNA use 20 μL total RNA) and add 80 gL RT reaction mix from step 5, 2, 3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and β-actin.

Following the synthesis of first strand cDNA, one particular embodiment of the approach for amplification of first strand cDNA by PCR, followed by detection and quantification of constituents of a Gene Expression Panel (Precision Profile™) is performed using the ABI Prism® 7900 Sequence Detection System as follows:

Materials

1. 20X Primer/Probe Mix for each gene of interest.

2. 20X Primer/Probe Mix for 18S endogenous control.

3. 2X Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism® 7700 or 7900 Sequence Detector.

Methods

1. Make stocks of each Primer/Probe mix containing the Primer/Probe for the gene of interest, Primer/Probe for 18S endogenous control, and 2×PCR Master Mix as follows. Make sufficient excess to allow for pipetting error e.g., approximately 10% excess. The following example illustrates a typical set up for one gene with quadruplicate samples testing two conditions (2 plates).

| | 1X (1 well) (μL) |
|---|---|
| 2X Master Mix | 7.5 |
| 20X 18S Primer/Probe Mix | 0.75 |
| 20X Gene of interest Primer/Probe Mix | 0.75 |
| Total | 9.0 |

2. Make stocks of cDNA targets by diluting 95 μL of cDNA into 2000 μL of water. The amount of cDNA is adjusted to give C_{T} values between 10 and 18, typically between 12 and 16.

3. Pipette 9 μL of Primer/Probe mix into the appropriate wells of an Applied Biosystems 384-Well Optical Reaction Plate.

4. Pipette 10 μL of cDNA stock solution into each well of the Applied Biosystems 384-Well Optical Reaction Plate.

5. Seal the plate with Applied Biosystems Optical Caps, or optical-clear film.

6. Analyze the plate on the ABI Prism® 7900 Sequence Detector.

In another embodiment of the invention, the use of the primer probe with the first strand cDNA as described above to permit measurement of constituents of a Gene Expression Panel (Precision Profile™) is performed using a QPCR assay on Cepheid SmartCycler® and GeneXpert® Instruments as follows:
I. To run a QPCR assay in duplicate on the Cepheid SmartCycler® instrument containing three target genes and one reference gene, the following procedure should be followed.

A. With 20× Primer/Probe Stocks.

Materials
1. SmartMix™-HM lyophilized Master Mix.
2. Molecular grade water.
3. 20X Primer/Probe Mix for the 18S endogenous control gene. The endogenous control gene will be dual labeled with VIC-MGB or equivalent.
4. 20X Primer/Probe Mix for each for target gene one, dual labeled with FAM-BHQ1 or equivalent.
5. 20X Primer/Probe Mix for each for target gene two, dual labeled with Texas Red-BHQ2 or equivalent.
6. 20X Primer/Probe Mix for each for target gene three, dual labeled with Alexa 647-BHQ3 or equivalent.
7. Tris buffer, pH 9.0
8. cDNA transcribed from RNA extracted from sample.
9. SmartCycler® 25 μL tube.
10. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| 20X 18S Primer/Probe Mix | 2.5 | μL |
| 20X Target Gene 1 Primer/Probe Mix | 2.5 | μL |
| 20X Target Gene 2 Primer/Probe Mix | 2.5 | μL |
| 20X Target Gene 3 Primer/Probe Mix | 2.5 | μL |
| Tris Buffer, pH 9.0 | 2.5 | μL |
| Sterile Water | 34.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene C_{T} value between 12 and 16.
3. Add 3 μL of the prepared cDNA sample to the reagent mixture bringing the total volume to 50 Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.

B. With Lyophilized SmartBeads™.

Materials
1. SmartMix™-HM lyophilized Master Mix.
2. Molecular grade water.
3. SmartBeads™ containing the 18S endogenous control gene dual labeled with VIC-MGB or equivalent, and the three target genes, one dual labeled with FAM-BHQ1 or equivalent, one dual labeled with Texas Red-BHQ2 or equivalent and one dual labeled with Alexa 647-BHQ3 or equivalent.
4. Tris buffer, pH 9.0
5. cDNA transcribed from RNA extracted from sample.
6. SmartCycler® 25 μL tube.
7. Cepheid SmartCycler® instrument.

Methods
1. For each cDNA sample to be investigated, add the following to a sterile 650 μL tube.

| | | |
|---|---|---|
| SmartMix ™-HM lyophilized Master Mix | 1 | bead |
| SmartBead ™ containing four primer/probe sets | 1 | bead |
| Tris Buffer, pH 9.0 | 2.5 | μL |
| Sterile Water | 44.5 | μL |
| Total | 47 | μL |

Vortex the mixture for 1 second three times to completely mix the reagents. Briefly centrifuge the tube after vortexing.
2. Dilute the cDNA sample so that a 3 μL addition to the reagent mixture above will give an 18S reference gene C_{T} value between 12 and 16.
3. Add 3 μL of the prepared cDNA sample to the reagent mixture bringing the total volume to 504. Vortex the mixture for 1 second three times to completely mix the reagents. Briefly centrifuge the tube after vortexing.
4. Add 25 μL of the mixture to each of two SmartCycler® tubes, cap the tube and spin for 5 seconds in a microcentrifuge having an adapter for SmartCycler® tubes.
5. Remove the two SmartCycler® tubes from the microcentrifuge and inspect for air bubbles. If bubbles are present, re-spin, otherwise, load the tubes into the SmartCycler® instrument.
6. Run the appropriate QPCR protocol on the SmartCycler®, export the data and analyze the results.
II. To run a QPCR assay on the Cepheid GeneXpert® instrument containing three target genes and one reference gene, the following procedure should be followed. Note that to do duplicates, two self contained cartridges need to be loaded and run on the GeneXpert® instrument.

Materials
1. Cepheid GeneXpert® self contained cartridge preloaded with a lyophilized SmartMix™-HM master mix bead and a lyophilized SmartBead™ containing four primer/probe sets.
2. Molecular grade water, containing Tris buffer, pH 9.0.
3. Extraction and purification reagents.
4. Clinical sample (whole blood, RNA, etc.)
5. Cepheid GeneXpert® instrument.

Methods
1. Remove appropriate GeneXpert® self contained cartridge from packaging.
2. Fill appropriate chamber of self contained cartridge with molecular grade water with Tris buffer, pH 9.0.
3. Fill appropriate chambers of self contained cartridge with extraction and purification reagents.
4. Load aliquot of clinical sample into appropriate chamber of self contained cartridge.
5. Seal cartridge and load into GeneXpert® instrument.
6. Run the appropriate extraction and amplification protocol on the GeneXpert® and analyze the resultant data.

In yet another embodiment of the invention, the use of the primer probe with the first strand cDNA as described above to permit measurement of constituents of a Gene Expression Panel (Precision Profile™) is performed using a QPCR assay on the Roche LightCycler® 480 Real-Time PCR System as follows:

Materials

1. 20X Primer/Probe stock for the 18S endogenous control gene. The endogenous control gene may be dual labeled with either VIC-MGB or VIC-TAMRA.

2. 20X Primer/Probe stock for each target gene, dual labeled with either FAM-TAMRA or FAM-BHQ1.

3. 2X LightCycler® 490 Probes Master (master mix).

4. 1X cDNA sample stocks transcribed from RNA extracted from samples.

5. 1X TE buffer, pH 8.0.

6. LightCycler® 480 384-well plates.

7. Source MDx 24 gene Precision Profile™ 96-well intermediate plates.

8. RNase/DNase free 96-well plate.

9. 1.5 mL microcentrifuge tubes.

10. Beckman/Coulter Biomek® 3000 Laboratory Automation Workstation.

11. Velocity11 Bravo™ Liquid Handling Platform.

12. LightCycler® 480 Real-Time PCR System.

Methods:

1. Remove a Source MDx 24 gene Precision Profile™ 96-well intermediate plate from the freezer, thaw and spin in a plate centrifuge.

2. Dilute four (4) 1× cDNA sample stocks in separate 15 mL microcentrifuge tubes with the total final volume for each of 540 μL.

3. Transfer the 4 diluted cDNA samples to an empty RNase/DNase free 96-well plate using the Biomek® 3000 Laboratory Automation Workstation.

4. Transfer the cDNA samples from the cDNA plate created in step 3 to the thawed and centrifuged Source MDx 24 gene Precision Profile™ 96-well intermediate plate using Biomek® 3000 Laboratory Automation Workstation. Seal the plate with a foil seal and spin in a plate centrifuge.

5. Transfer the contents of the cDNA-loaded Source MDx 24 gene Precision Profile™ 96-well intermediate plate to a new LightCycler® 480 384-well plate using the Bravo™ Liquid Handling Platform. Seal the 384-well plate with a LightCycler® 480 optical sealing foil and spin in a plate centrifuge for 1 minute at 2000 rpm.

6. Place the sealed in a dark 4° C. refrigerator for a minimum of 4 minutes.

7. Load the plate into the LightCycler® 480 Real-Time PCR System and start the LightCycler® 480 software. Chose the appropriate run parameters and start the run.

8. At the conclusion of the run, analyze the data and export the resulting CP values to the database.

In some instances, target gene FAM measurements may be beyond the detection limit of the particular platform instrument used to detect and quantify constituents of a Gene Expression Panel (Precision Profile™). To address the issue of “undetermined” gene expression measures as lack of expression for a particular gene, the detection limit may be reset and the “undetermined” constituents may be “flagged”. For example without limitation, the ABI Prism® 7900HT Sequence Detection System reports target gene FAM measurements that are beyond the detection limit of the instrument (>40 cycles) as “undetermined”. Detection Limit Reset is performed when at least 1 of 3 target gene FAM C_{T} replicates are not detected after 40 cycles and are designated as “undetermined”. “Undetermined” target gene FAM C_{T} replicates are re-set to 40 and flagged. C_{T} normalization (Δ C_{T}) and relative expression calculations that have used re-set FAM C_{T} values are also flagged.

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition, e.g., ocular disease. The concept of biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, and/or a particular agent.

The choice of a baseline profile data set for creating a calibrated profile data set is related to the biological condition to be evaluated, monitored, or predicted, as well as, the intended use of the calibrated panel, e.g., as to monitor drug development, quality control or other uses. It may be desirable to access baseline profile data sets from the same subject for whom a first profile data set is obtained or from different subject at varying times, exposures to stimuli, drugs or complex compounds; or may be derived from like or dissimilar populations or sets of subjects. The baseline profile data set may be normal, healthy baseline.

The profile data set may arise from the same subject for which the first data set is obtained, where the sample is taken at a separate or similar time, a different or similar site or in a different or similar biological condition. For example, a sample may be taken before stimulation or after stimulation with an exogenous compound or substance, such as before or after therapeutic treatment. Alternatively the sample is taken before or include before or after a surgical procedure for ocular disease. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library along with or separate from the baseline profile data base and optionally the first profile data set although the first profile data set would normally become incorporated into a baseline profile data set under suitable classification criteria. The remarkable consistency of Gene Expression Profiles associated with a given biological condition makes it valuable to store profile data, which can be used, among other things for normative reference purposes. The normative reference can serve to indicate the degree to which a subject conforms to a given biological condition (healthy or diseased) and, alternatively or in addition, to provide a target for clinical intervention.

Selected baseline profile data sets may be also be used as a standard by which to judge manufacturing lots in terms of efficacy, toxicity, etc. Where the effect of a therapeutic agent is being measured, the baseline data set may correspond to Gene Expression Profiles taken before administration of the agent. Where quality control for a newly manufactured product is being determined, the baseline data set may correspond with a gold standard for that product. However, any suitable normalization techniques may be employed. For example, an average baseline profile data set is obtained from authentic material of a naturally grown herbal nutraceutical and compared over time and over different lots in order to demonstrate consistency, or lack of consistency, in lots of compounds prepared for release.

### Calibrated Data

Given the repeatability achieved in measurement of gene expression, described above in connection with “Gene Expression Panels” (Precision Profiles™) and “gene amplification”, it was concluded that where differences occur in measurement under such conditions, the differences are attributable to differences in biological condition. Thus, it has been found that calibrated profile data sets are highly reproducible in samples taken from the same individual under the same conditions. Similarly, it has been found that calibrated profile data sets are reproducible in samples that are repeatedly tested. Also found have been repeated instances wherein calibrated profile data sets obtained when samples from a subject are exposed ex vivo to a compound are comparable to calibrated profile data from a sample that has been exposed to a sample in vivo.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within 20%, and typically within 10%. In accordance with embodiments of the invention, a pattern of increasing, decreasing and no change in relative gene expression from each of a plurality of gene loci examined in the Gene Expression Panel (Precision Profile™) may be used to prepare a calibrated profile set that is informative with regards to a biological condition, biological efficacy of an agent treatment conditions or for comparison to populations or sets of subjects or samples, or for comparison to populations of cells. Patterns of this nature may be used to identify likely candidates for a drug trial, used alone or in combination with other clinical indicators to be diagnostic or prognostic with respect to a biological condition or may be used to guide the development of a pharmaceutical or nutraceutical through manufacture, testing and marketing.

The numerical data obtained from quantitative gene expression and numerical data from calibrated gene expression relative to a baseline profile data set may be stored in databases or digital storage mediums and may be retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites.

The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the ocular disease or conditions related to ocular disease to be evaluated, with the calibrated profile data set being a comparison between the first profile data set and the baseline profile data set, thereby providing evaluation of ocular disease or conditions related to ocular disease of the subject.

In yet other embodiments, the function is a mathematical function and is other than a simple difference, including a second function of the ratio of the corresponding member of first profile data set to the corresponding member of the baseline profile data set, or a logarithmic function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

In other embodiments, a clinical indicator may be used to assess the ocular disease or conditions related to ocular disease of the relevant set of subjects by interpreting the calibrated profile data set in the context of at least one other clinical indicator, wherein the at least one other clinical indicator is selected from the group consisting of blood chemistry, X-ray or other radiological or metabolic imaging technique, molecular markers in the blood (e.g., carcinoembryonic antigen, CA19-9, and C-Reactive Protein (CRP)), other chemical assays, and physical findings.

### Index Construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel (Precision Profile™) giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of a biological condition.

An index may be constructed using an index function that maps values in a Gene Expression Profile into a single value that is pertinent to the biological condition at hand. The values in a Gene Expression Profile are the amounts of each constituent of the Gene Expression Panel (Precision Profile™). These constituent amounts form a profile data set, and the index function generates a single value—the index—from the members of the profile data set.

The index function may conveniently be constructed as a linear sum of terms, each term being what is referred to herein as a “contribution function” of a member of the profile data set. For example, the contribution function may be a constant times a power of a member of the profile data set. So the index function would have the form

I=ΣCiMi^{P(i)},

where I is the index, Mi is the value of the member i of the profile data set, Ci is a constant, and P(i) is a power to which Mi is raised, the sum being formed for all integral values of i up to the number of members in the data set. We thus have a linear polynomial expression. The role of the coefficient Ci for a particular gene expression specifies whether a higher ΔC_{T} value for this gene either increases (a positive Ci) or decreases (a lower value) the likelihood of ocular disease, the ΔC_{T} values of all other genes in the expression being held constant.

The values Ci and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Massachusetts, called Latent Gold®. Alternatively, other simpler modeling techniques may be employed in a manner known in the art. The index function for ocular disease may be constructed, for example, in a manner that a greater degree of ocular disease (as determined by the profile data set for any of the Precision Profiles™ described herein (Tables 1-2)) correlates with a large value of the index function. As discussed in further detail below, a meaningful ocular disease index that is proportional to the expression, was constructed as follows:

7.479+0.2447{SERPINB2}−{TGFB1}

where the braces around a constituent designate measurement of such constituent and the constituents are a subset of the Precision Profile™ for Ocular Disease included in Table 1A and 1B or Precision Profile™ for Inflammatory Response shown in Table 2.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

As an example, the index can be constructed, in relation to a normative Gene Expression Profile for a population or set of healthy subjects, in such a way that a reading of approximately 1 characterizes normative Gene Expression Profiles of healthy subjects. Let us further assume that the biological condition that is the subject of the index is ocular disease; a reading of 1 in this example thus corresponds to a Gene Expression Profile that matches the norm for healthy subjects. A substantially higher reading then may identify a subject experiencing ocular disease, or a condition related to ocular disease. The use of 1 as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since it was determined that Gene Expression Profile values (and accordingly constructed indices based on them) tend to be normally distributed, the O-centered index constructed in this manner is highly informative. It therefore facilitates use of the index in diagnosis of disease and setting objectives for treatment.

Still another embodiment is a method of providing an index pertinent to ocular disease or conditions related to ocular disease of a subject based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the presumptive signs of ocular disease, the panel including at least two of the constituents of any of the genes listed in the Precision Profiles described herein (listed in Tables 1-2). In deriving the profile data set, such measure for each constituent is achieved under measurement conditions that are substantially repeatable, at least one measure from the profile data set is applied to an index function that provides a mapping from at least one measure of the profile data set into one measure of the presumptive signs of ocular disease, so as to produce an index pertinent to the ocular disease or conditions related to ocular disease of the subject.

As another embodiment of the invention, an index function I of the form

I=C₀+ΣCiM₁ᵢ^{P1(i)}M₂ᵢ^{P2(i)},

can be employed, where M₁ and M₂ are values of the member i of the profile data set, Cᵢ is a constant determined without reference to the profile data set, and P1 and P2 are powers to which M₁ and M₂ are raised. The role of P1(i) and P2(i) is to specify the specific functional form of the quadratic expression, whether in fact the equation is linear, quadratic, contains cross-product terms, or is constant. For example, when P1=P2=0, the index function is simply the sum of constants; when P1=1 and P2=0, the index function is a linear expression; when P1=P2=1, the index function is a quadratic expression.

The constant C₀ serves to calibrate this expression to the biological population of interest that is characterized by having ocular disease. In this embodiment, when the index value equals 0, the odds are 50:50 of the subject having ocular disease vs a normal subject. More generally, the predicted odds of the subject having ocular disease is [exp(Iᵢ)], and therefore the predicted probability of having ocular disease is [exp(Iᵢ)]/[1+exp((Iᵢ)]. Thus, when the index exceeds 0, the predicted probability that a subject has ocular disease is higher than 0.5, and when it falls below 0, the predicted probability is less than 0.5.

The value of C₀ may be adjusted to reflect the prior probability of being in this population based on known exogenous risk factors for the subject. In an embodiment where C₀ is adjusted as a function of the subject's risk factors, where the subject has prior probability pᵢ of having ocular disease based on such risk factors, the adjustment is made by increasing (decreasing) the unadjusted C₀ value by adding to C₀ the natural logarithm of the following ratio: the prior odds of having ocular disease taking into account the risk factors/the overall prior odds of having ocular disease without taking into account the risk factors.

### Performance and Accuracy Measures of the Invention

The performance and thus absolute and relative clinical usefulness of the invention may be assessed in multiple ways as noted above. Amongst the various assessments of performance, the invention is intended to provide accuracy in clinical diagnosis and prognosis. The accuracy of a diagnostic or prognostic test, assay, or method concerns the ability of the test, assay, or method to distinguish between subjects having ocular disease is based on whether the subjects have an “effective amount” or a “significant alteration” in the levels of an ocular disease associated gene. By “effective amount” or “significant alteration”, it is meant that the measurement of an appropriate number of ocular disease associated gene (which may be one or more) is different than the predetermined cut-off point (or threshold value) for that ocular disease associated gene and therefore indicates that the subject has ocular disease for which the ocular disease associated gene(s) is a determinant.

The difference in the level of ocular disease associated gene(s) between normal and abnormal is preferably statistically significant. As noted below, and without any limitation of the invention, achieving statistical significance, and thus the preferred analytical and clinical accuracy, generally but not always requires that combinations of several ocular disease associated gene(s) be used together in panels and combined with mathematical algorithms in order to achieve a statistically significant ocular disease associated gene index.

In the categorical diagnosis of a disease state, changing the cut point or threshold value of a test (or assay) usually changes the sensitivity and specificity, but in a qualitatively inverse relationship. Therefore, in assessing the accuracy and usefulness of a proposed medical test, assay, or method for assessing a subject's condition, one should always take both sensitivity and specificity into account and be mindful of what the cut point is at which the sensitivity and specificity are being reported because sensitivity and specificity may vary significantly over the range of cut points. Use of statistics such as AUC, encompassing all potential cut point values, is preferred for most categorical risk measures using the invention, while for continuous risk measures, statistics of goodness-of-fit and calibration to observed results or other gold standards, are preferred.

Using such statistics, an “acceptable degree of diagnostic accuracy”, is herein defined as a test or assay (such as the test of the invention for determining an effective amount or a significant alteration of ocular disease associated gene(s), which thereby indicates the presence of a ocular disease in which the AUC (area under the ROC curve for the test or assay) is at least 0.60, desirably at least 0.65, more desirably at least 0.70, preferably at least 0.75, more preferably at least 0.80, and most preferably at least 0.85.

By a “very high degree of diagnostic accuracy”, it is meant a test or assay in which the AUC (area under the ROC curve for the test or assay) is at least 0.75, desirably at least 0.775, more desirably at least 0.800, preferably at least 0.825, more preferably at least 0.850, and most preferably at least 0.875.

The predictive value of any test depends on the sensitivity and specificity of the test, and on the prevalence of the condition in the population being tested. This notion, based on Bayes' theorem, provides that the greater the likelihood that the condition being screened for is present in an individual or in the population (pre-test probability), the greater the validity of a positive test and the greater the likelihood that the result is a true positive. Thus, the problem with using a test in any population where there is a low likelihood of the condition being present is that a positive result has limited value (i.e., more likely to be a false positive). Similarly, in populations at very high risk, a negative test result is more likely to be a false negative.

As a result, ROC and AUC can be misleading as to the clinical utility of a test in low disease prevalence tested populations (defined as those with less than 1% rate of occurrences (incidence) per annum, or less than 10% cumulative prevalence over a specified time horizon). Alternatively, absolute risk and relative risk ratios as defined elsewhere in this disclosure can be employed to determine the degree of clinical utility. Populations of subjects to be tested can also be categorized into quartiles by the test's measurement values, where the top quartile (25% of the population) comprises the group of subjects with the highest relative risk for developing ocular disease, and the bottom quartile comprising the group of subjects having the lowest relative risk for developing ocular disease. Generally, values derived from tests or assays having over 2.5 times the relative risk from top to bottom quartile in a low prevalence population are considered to have a “high degree of diagnostic accuracy,” and those with five to seven times the relative risk for each quartile are considered to have a “very high degree of diagnostic accuracy.” Nonetheless, values derived from tests or assays having only 1.2 to 2.5 times the relative risk for to each quartile remain clinically useful are widely used as risk factors for a disease. Often such lower diagnostic accuracy tests must be combined with additional parameters in order to derive meaningful clinical thresholds for therapeutic intervention, as is done with the aforementioned global risk assessment indices.

A health economic utility function is yet another means of measuring the performance and clinical value of a given test, consisting of weighting the potential categorical test outcomes based on actual measures of clinical and economic value for each. Health economic performance is closely related to accuracy, as a health economic utility function specifically assigns an economic value for the benefits of correct classification and the costs of misclassification of tested subjects. As a performance measure, it is not unusual to require a test to achieve a level of performance which results in an increase in health economic value per test (prior to testing costs) in excess of the target price of the test.

In general, alternative methods of determining diagnostic accuracy are commonly used for continuous measures, when a disease category or risk category (such as those at risk for having a bone fracture) has not yet been clearly defined by the relevant medical societies and practice of medicine, where thresholds for therapeutic use are not yet established, or where there is no existing gold standard for diagnosis of the pre-disease. For continuous measures of risk, measures of diagnostic accuracy for a calculated index are typically based on curve fit and calibration between the predicted continuous value and the actual observed values (or a historical index calculated value) and utilize measures such as R squared, Hosmer-Lemeshow P-value statistics and confidence intervals. It is not unusual for predicted values using such algorithms to be reported including a confidence interval (usually 90% or 95% CI) based on a historical observed cohort's predictions, as in the test for risk of future breast cancer recurrence commercialized by Genomic Health, Inc. (Redwood City, Calif.).

In general, by defining the degree of diagnostic accuracy, i.e., cut points on a ROC curve, defining an acceptable AUC value, and determining the acceptable ranges in relative concentration of what constitutes an effective amount of the ocular disease associated gene(s) of the invention allows for one of skill in the art to use the ocular disease associated gene(s) to identify, diagnose, or prognose subjects with a pre-determined level of predictability and performance.

Results from the ocular disease associated gene(s) indices thus derived can then be validated through their calibration with actual results, that is, by comparing the predicted versus observed rate of disease in a given population, and the best predictive ocular disease associated gene(s) selected for and optimized through mathematical models of increased complexity. Many such formula may be used; beyond the simple non-linear transformations, such as logistic regression, of particular interest in this use of the present invention are structural and synactic classification algorithms, and methods of risk index construction, utilizing pattern recognition features, including established techniques such as the Kth-Nearest Neighbor, Boosting, Decision Trees, Neural Networks, Bayesian Networks, Support Vector Machines, and Hidden Markov Models, as well as other formula described herein.

Furthermore, the application of such techniques to panels of multiple ocular disease associated gene(s) is provided, as is the use of such combination to create single numerical “risk indices” or “risk scores” encompassing information from multiple ocular disease associated gene(s) inputs. Individual B ocular disease associated gene(s) may also be included or excluded in the panel of ocular disease associated gene(s) used in the calculation of the ocular disease associated gene(s) indices so derived above, based on various measures of relative performance and calibration in validation, and employing through repetitive training methods such as forward, reverse, and stepwise selection, as well as with genetic algorithm approaches, with or without the use of constraints on the complexity of the resulting ocular disease associated gene(s) indices.

The above measurements of diagnostic accuracy for ocular disease associated gene(s) are only a few of the possible measurements of the clinical performance of the invention. It should be noted that the appropriateness of one measurement of clinical accuracy or another will vary based upon the clinical application, the population tested, and the clinical consequences of any potential misclassification of subjects. Other important aspects of the clinical and overall performance of the invention include the selection of ocular disease associated gene(s) so as to reduce overall ocular disease associated gene(s) variability (whether due to method (analytical) or biological (pre-analytical variability, for example, as in diurnal variation), or to the integration and analysis of results (post-analytical variability) into indices and cut-off ranges), to assess analyte stability or sample integrity, or to allow the use of differing sample matrices amongst blood, cells, serum, plasma, urine, etc.

### Kits

The invention also includes a ocular disease detection reagent, i.e., nucleic acids that specifically identify one or more ocular disease or condition related to ocular disease nucleic acids (e.g., any gene listed in Tables 1-5, 7-9, and 11-13, and angiogenesis genes; sometimes referred to herein as ocular disease associated genes or ocular disease associated constituents) by having homologous nucleic acid sequences, such as oligonucleotide sequences, complementary to a portion of the ocular disease genes nucleic acids or antibodies to proteins encoded by the ocular disease genes nucleic acids packaged together in the form of a kit. The oligonucleotides can be fragments of the ocular disease genes. For example the oligonucleotides can be 200, 150, 100, 50, 25, 10 or less nucleotides in length. The kit may contain in separate containers a nucleic acid or antibody (either already bound to a solid matrix or packaged separately with reagents for binding them to the matrix), control formulations (positive and/or negative), and/or a detectable label. Instructions (i.e., written, tape, VCR, CD-ROM, etc.) for carrying out the assay may be included in the kit. The assay may for example be in the form of PCR, a Northern hybridization or a sandwich ELISA, as known in the art.

For example, ocular disease genes detection reagents can be immobilized on a solid matrix such as a porous strip to form at least one ocular disease associated gene detection site. The measurement or detection region of the porous strip may include a plurality of sites containing a nucleic acid. A test strip may also contain sites for negative and/or positive controls. Alternatively, control sites can be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of ocular disease genes present in the sample. The detection sites may be configured in any suitably detectable shape and are typically in the shape of a bar or dot spanning the width of a test strip.

Alternatively, ocular disease detection genes can be labeled (e.g., with one or more fluorescent dyes) and immobilized on lyophilized beads to form at least one ocular disease associated gene detection site. The beads may also contain sites for negative and/or positive controls. Upon addition of the test sample, the number of sites displaying a detectable signal provides a quantitative indication of the amount of ocular disease genes present in the sample.

Alternatively, the kit contains a nucleic acid substrate array comprising one or more nucleic acid sequences. The nucleic acids on the array specifically identify one or more nucleic acid sequences represented by ocular disease genes (see Tables 1-5, 7-9, and 11-13). In various embodiments, the expression of 1, 2, 3, 4, 5, 6, 7, 8, 9, 10, 15, 20, 25, 40 or 50 or more of the sequences represented by ocular disease genes (see Tables 1-5, 7-9, and 11-13) can be identified by virtue of binding to the array. The substrate array can be on, i.e., a solid substrate, i.e., a “chip” as described in U.S. Pat. No. 5,744,305. Alternatively, the substrate array can be a solution array, i.e., Luminex, Cyvera, Vitra and Quantum Dots' Mosaic.

The skilled artisan can routinely make antibodies, nucleic acid probes, i.e., oligonucleotides, aptamers, siRNAs, antisense oligonucleotides, against any of the ocular disease genes listed in Tables 1-5, 7-9, and 11-13.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### EXAMPLES

### Example 1

### Normal Pressure Glaucoma Clinical Data Analyzed with Latent Class Modeling (1-Gene and 2-Gene Models) Based on The Precision Profile™ for Ocular Disease

RNA was isolated using the PAXgene™ System from blood samples obtained from a total of 17 subjects suffering from normal pressure glaucoma (NPG) and 24 normal subjects.

From a targeted 96-gene Precision Profile™ for Ocular Disease (included in Table 1A), selected to be informative relative to biological state of ocular disease patients, primers and probes were prepared. Each of these genes was evaluated for significance (i.e., p-value) regarding their ability to discriminate between subjects afflicted with NPG and normal subjects. A ranking of the top 96 genes is shown in Tables 3 and 4, summarizing the results of significance tests for the difference in the mean expression levels for normal subjects and subjects suffering from NPG. Since competing methods are available that are justified under different assumptions, the p-values were computed in 2 different ways:
1) Based on 1-way ANOVA. This approach assumes that the gene expression is normally distributed with the same variance within each of the 2 populations (Table 3).
2) Based on stepwise logistic regression (STEP analysis), where group membership (Normal vs. NPG) is predicted as a function of the gene expression (Table 4). Conceptually, this is the reverse of what is done in the ANOVA approach where the gene expression is predicted as a function of the group. The logistic distribution holds true under several different distributional assumptions, including those that are made in the 1-way ANOVA approach.

Thus, this second strategy is justified under a more general class of distributional assumptions than the ANOVA approach.

As expected, the two different approaches yield comparable p-values and comparable rankings for the genes. As can be seen from Tables 3 and 4, the p-values are fairly similar for most genes except those having extremely low p-values, which include some of the low-expressing genes (i.e., instances where target gene FAM measurements were beyond the detection limit (i.e., very high ΔC_{T} values which indicate low expression) of the particular platform instrument used to detect and quantify constituents of a Gene Expression Panel (Precision Profile™)). To address the issue of “undetermined” gene expression measures as lack of expression for a particular gene, the detection limit was reset and the “undetermined” constituents were “flagged”, as previously described. C_{T} normalization (Δ C_{T}) and relative expression calculations that have used re-set FAM C_{T} values were also flagged. These low expressing genes (i.e., re-set FAM C_{T} values) were eliminated from the analysis if 50% or more ΔC_{T} values from either of the 2 groups were flagged. Although such genes were eliminated from the statistical analyses described herein, one skilled in the art would recognize that such genes may play a relevant role in ocular disease.

Low-expressing genes which were excluded from the gene models are shown shaded gray in Tables 3 and 4). Strong predictive results were obtained without using the genes, as described below.

After excluding the under-expressing genes, the gene TGFB1 and was found to be significant at the 0.05 level using both the 1-WAY ANOVA or STEP analysis and was subject to further stepwise logistic regression analysis (described below), to generate gene models capable of correctly classifying NPG and normal subjects with at least 75% accuracy, as described in Table 5 below. As demonstrated in Table 5, as few as one gene allowed for discrimination between individuals with NPG and normals at an accuracy of at least 75%.

Gene Expression Modeling

Gene expression profiles were obtained using the 96 gene expression panel from Table 1A, and the Search procedure in GOLDMineR (Magidson, 1998) to implement stepwise logistic regressions (STEP analysis) for predicting the dichotomous variable that distinguishes subjects suffering from NPG from normal subjects as a function of the 96 genes (ranked in Tables 3 and 4). The STEP analysis was performed under the assumption that the gene expressions follow a multinormal distribution, with different means and different variance-covariance matrices for the normal and NPG population.

TGFB1

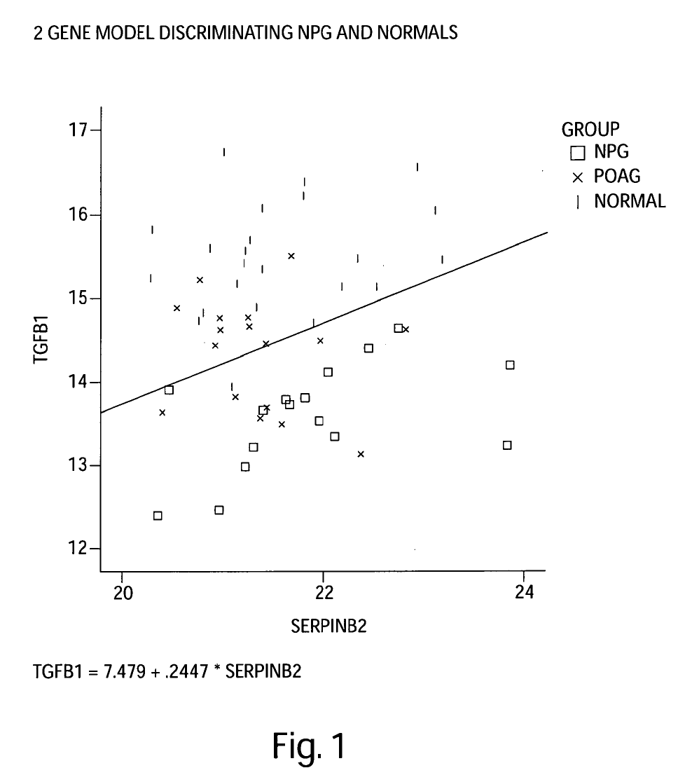

As can be seen from Table 5, Gene 1 column, the classification rate computed for normal v. NPG subjects using TGFB1 alone met the 75% criteria. TGFB1 alone was capable of distinguishing between NPG subjects with 100% accuracy, and normal subjects with 92% accuracy. TGFB1 was subject to a further analysis in a 2 gene model where all 95 remaining genes were evaluated as the second gene in this 2-gene model. All models that yielded significant incremental p-values, at the 0.05 level, for the second gene were then analyzed using Latent Gold to find R² values. The R² statistic is a less formal statistical measure of goodness of prediction, which varies between 0 (predicted probability of having NPG is constant regardless of ΔC_{T} values on the 2 genes) to 1 (predicted probability of having NPG=1 for each NPG subject, and =0 for each Normal subject). If the 2-gene model yielded an R² value greater than 0.6 it was kept as a model that discriminated well. If these models met the 0.6 cutoff, their statistical output from Latent Gold, was then used to determine classification percentages. As can be seen from Table 5, Gene 2 column, the 2-gene model TGFB1 and SERPINB2 correctly classified subjects suffering from NPG or normal subjects with 100% and 92% accuracy, respectively. These results are depicted graphically in FIG. 1.

FIG. 1 shows that a line can almost perfectly distinguish the two groups using the 2 gene model TGFB1 and SERPINB2. This discrimination line is an example of the Index Function evaluated at a particular logit (log odds) value. Values above and to the left of the line are predicted to be in the normal, those below and to the right of the line in the NPG population. This is a simplified version of the “Index function” as displayed in two dimensions, where the gene with positive coefficients (positive contributions) (SERPINB2) is plotted along the horizontal axis, and the gene with negative coefficients (TGFB1) is plotted along the vertical axis. ‘Positive’ coefficients means that the higher the ΔC_{T} values for those genes (holding the other genes constant) increases the predicted logit, and thus the predicted probability of being in the diseased group.

The intercept (alpha) and slope (beta) of the discrimination line was computed according to the data shown in Table 6. A cutoff of 0.3289 was used to compute alpha (equals −0.7131644 in logit units).

The following equation is given below the graph shown in FIG. 1:

Normal Pressure Glaucoma Discrimination Line: TGFB1=7.479+0.2447*SERPINEB2.

Subjects below and to the right of this discrimination line have a predicted probability of being in the diseased group higher than the cutoff probability of 0.3289.

The intercept C₀=7.479 was computed by taking the difference between the intercepts for the 2 groups [34.3695−(−34.3695)=68.739] and subtracting the log-odds of the cutoff probability (−0.7131644). This quantity was then multiplied by −1/X where X is the coefficient for TGFB1 (−9.2861).

### Example 2

### Primary Open Angle Glaucoma Clinical Data Analyzed with Latent Class Modeling (1-Gene and 2-Gene Models) Based on The Precision Profile™ for Ocular Disease

RNA was isolated using the PAXgene™ System from blood samples obtained from a total of 17 subjects suffering from primary open angle glaucoma (POAG) and 24 normal subjects.

The 96 genes of the gene expression panel from Table 1A as described above were evaluated for significance (i.e., p-value) regarding their ability to discriminate between subjects afflicted with POAG and normal subjects. The p-values were computed using the 1-way ANOVA approach and stepwise logistic regression (STEP analysis) as described in Example 1. A ranking of the top 96 genes is shown in Table 7 (1-way ANOVA approach) and Table 8 (STEP analysis), summarizing the results of significance tests for the difference in the mean expression levels for normal subjects and subjects suffering from POAG.

As expected, the two different approaches yield comparable p-values and comparable rankings for the genes. As can be seen from Tables 7 and 8, the p-values are fairly similar for most genes except those having extremely low p-values, which include some low-expressing genes. Low-expressing genes (previously described, shown shaded gray in Tables 7 and 8) were excluded from the gene models. Strong predictive results were obtained without using the genes, as described below.

After excluding the low-expressing genes, the MMP19 and was found to be significant at the 0.05 level using both the 1-WAY ANOVA approach or STEP analysis, and was subject to further stepwise logistic regression analysis (described below), to generate a multi-gene model capable of correctly classifying POAG and normal subjects with at least 75% accuracy, as described in Table 9 below. As demonstrated in Table 9, as few as one gene allowed for discrimination between individuals with NPG and normals at an accuracy of at least 75%.

Gene Expression Modeling

Gene expression profiles were obtained using the 96-gene panel from Table 1A and the Search procedure in GOLDMineR (Magidson, 1998) to implement stepwise logistic regressions (STEP analysis) for predicting the dichotomous variable that distinguishes subjects suffering from POAG from normal subjects as a function of the 96 genes (ranked in Tables 7 and 8). The STEP analysis was performed under the assumption that the gene expressions follow a multinormal distribution, with different means and different variance-covariance matrices for the normal and POAG population.

Table 9, columns 1-2 show the maximized and adjusted classification rates for each multi-gene model. The ‘maximum overall rate’ is based on the predicted logit (predicted probability) cutoff that minimizes the total number of misclassifications in the sample. The ‘adjusted’ rate adjusts for different sample sizes in each group, maximizing the ‘equalized classification rate’ and thus tends to equalize the percentage classified correctly in each group. For example, suppose that there are 110 POAG subjects in the sample and only 50 normal subjects, and suppose that the adjusted rate was 90% for each group. This yields 11 misclassifications among the POAG subjects and 5 among the normals, a total of 16 misclassifications (overall, 90% correctly classified). By choosing a lower cutoff, more subjects are predicted to be in the POAG group, and fewer in the normal group; thus, more normal subjects will be misclassified. Suppose that with a lower cutoff, 2 fewer POAG subjects are misclassified at the cost of misclassifying 1 additional normal. Now, the correct classification rate for POAG subjects increases to 101/110=91.8% and the corresponding rate for normals reduces to 44/50=88%.

Overall, since the total number misclassified is reduced, the overall correct classification rate improves from 90% to 145/160=90.6%. However, weighting each group equally, the ‘equalized classification rate’ gets worse (91.8%+88%)/2=89.9%. The optimal cutoff on the ΔC_{T} value for each gene was chosen that maximized the overall correct classification rate. The actual correct classification rate for the POAG and normal subjects was computed based on this cutoff and determined as to whether both reached the 75% criteria.

MMP19

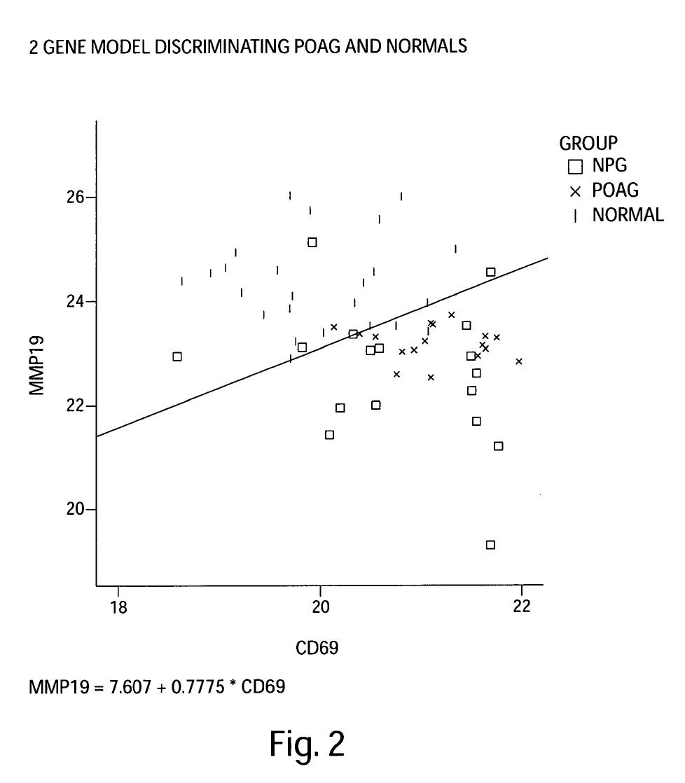

As can be seen from Table 9, Gene 1 column, the classification rate computed for normal v. POAG subjects using MMP19 alone met the 75% criteria. MMP19 alone was capable of distinguishing between POAG subjects with an adjusted rate of 82% accuracy, and normal subjects with 83% accuracy. MMP19 was subject to a further analysis in a 2 gene model where all 95 remaining genes were evaluated as the second gene in this 2-gene model. All models that yielded significant incremental p-values, at the 0.05 level, for the second gene were then analyzed using Latent Gold to find R² values. The R² statistic is a less formal statistical measure of goodness of prediction, which varies between 0 (predicted probability of having POAG is constant regardless of ΔC_{T} values on the 2 genes) to 1 (predicted probability of having POAG=1 for each POAG subject, and =0 for each Normal subject). If the 2-gene model yielded an R² value greater than 0.6 it was kept as a model that discriminated well. If these models met the 0.6 cutoff, their statistical output from Latent Gold, was then used to determine classification percentages. As can be seen from Table 9, Gene 2 column, the 2-gene model MMP19 and CD69 correctly classified subjects suffering from POAG or normal subjects with and adjusted 94% and 92% accuracy, respectively. These results are depicted graphically in FIG. 2.

FIG. 2 also shows that a line can almost perfectly distinguish the two groups using the 2 to gene model MMP19 and CD69. This discrimination line is an example of the Index Function evaluated at a particular logit (log odds) value. Values above and to the left of the line are predicted to be in the normal, those below and to the right in the POAG population. This is a simplified version of the “Index function” as displayed in two dimensions, where the gene with positive coefficients (positive contributions) (CD69) is plotted along the horizontal axis, and the gene with negative coefficients (MMP19) is plotted along the vertical axis. ‘Positive’ coefficients means that the higher the ΔC_{T} values for those genes (holding the other genes constant) increases the predicted logit, and thus the predicted probability of being in the diseased group.

The intercept (alpha) and slope (beta) of the discrimination line was computed according to the data shown in Table 10. A cutoff of 0.4149 was used to compute alpha (equals −0.343745 in logit units).

The following equation is given below the graph shown in FIG. 2:

Primary Open Angle Glaucoma Discrimination Line: MMP19=7.607+0.7775*CD69.

Subjects below and to the right of this discrimination line have a predicted probability of being in the diseased group higher than the cutoff probability of 0.4149.

The intercept C₀=7.606757 was computed by taking the difference between the intercepts for the 2 groups [13.1932−(−13.1932)=28.3864] and subtracting the log-odds of the cutoff probability (−0.343745). This quantity was then multiplied by −1/X where X is the coefficient for MMP19 (−3.514).

### Example 3

### Combined Primary Open Angle Glaucoma and Normal Pressure Glaucoma Clinical Data Analyzed with Latent Class Modeling (1-Gene and 2-Gene Models) Based on The Precision Profile™ for Ocular Disease

The gene expression data generated from the NPG and POAG studies described above in Examples 1 and 2 respectively, were combined and the Search procedure in GOLDMineR (Magidson, 1998) was used to implement stepwise logistic regressions (STEP analysis) for predicting the dichotomous variable capable of distinguishing subjects suffering from NPG or POAG from normal subjects as a function of the 96 genes.

The 96 genes of the gene expression panel from Table 1A as described above were evaluated for significance (i.e., p-value) regarding their ability to discriminate between subjects afflicted with NPG and POAG from normal subjects. The p-values were computed using the 1-way ANOVA approach and stepwise logistic regression (STEP analysis) as described in Example 1. A ranking of the top 96 genes is shown in Table 11 (1-way ANOVA approach) and Table 12 (STEP analysis), summarizing the results of significance tests for the difference in the mean expression levels for normal subjects and subjects suffering from NPG and POAG.

As expected, the two different approaches yield comparable p-values and comparable rankings for the genes. As can be seen from Tables 11 and 12, the p-values are fairly similar for most genes except those having extremely low p-values, which include some low-expressing genes. Low-expressing genes (previously described, shown shaded gray in Tables 11 and 12) were eliminated from the analysis as previously described. After excluding the low-expressing genes, TGFB1 and was found to be significant at the 0.05 level using both the 1-WAY ANOVA approach or STEP analysis, and was subject to further stepwise logistic regression analysis (described below), to generate a multi-gene model capable of correctly classifying NPG and POAG subjects from normal subjects with at least 75% accuracy, as described in Table 13 below. As demonstrated in Table 13, as few as one gene allowed for discrimination between individuals with NPG and POAG from normals with at least 75% accuracy.

The STEP analysis was performed under the assumption that the gene expressions follow a multinormal distribution, with different means and different variance-covariance matrices for the normal, NPG and POAG populations. Maximum and/or adjusted classification rates for the gene expression models identified were calculated as previously described in Example 2.

### TGFB1

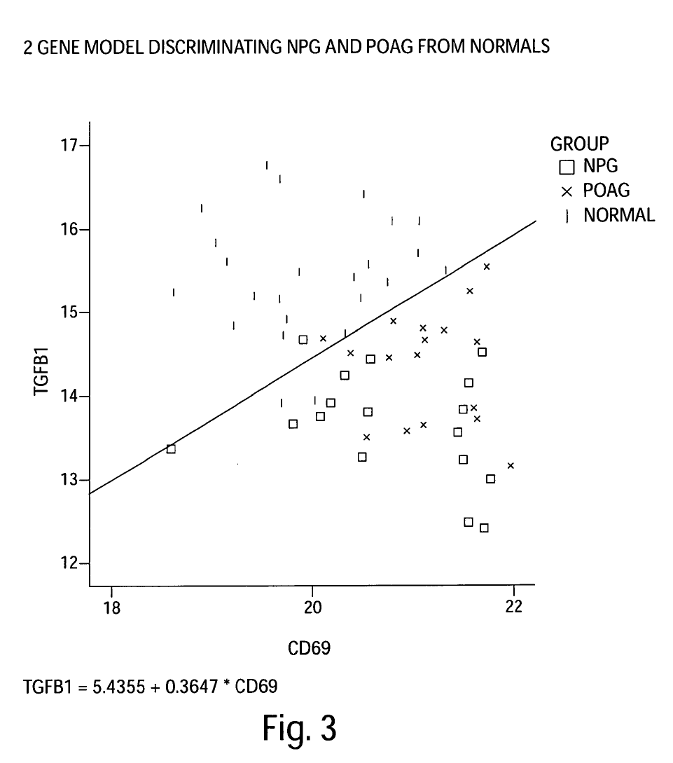

As can be seen from Table 13, Gene 1 column, the adjusted classification rate computed for normal v. combined NPG and POAG subjects using TGFB1 alone met the 75% criteria. TGFB1 alone was capable of distinguishing between NPG and POAG subjects with an adjusted rate of 85% accuracy, and normal subjects with 92% accuracy. TGFB1 was subject to a further analysis in a 2 gene model where all 95 remaining genes were evaluated as the second gene in this 2-gene model. All models that yielded significant incremental p-values, at the 0.05 level, for the second gene were then analyzed using Latent Gold to find R² values. The R² statistic is a less formal statistical measure of goodness of prediction, which varies between 0 (predicted probability of having NPG and POAG is constant regardless of ΔC_{T} values on the 2 genes) to 1 (predicted probability of having NPG and POAG=1 for each NPG and POAG subject, and =0 for each Normal subject). If the 2-gene model yielded an R² value greater than 0.6 it was kept as a model that discriminated well. If these models met the 0.6 cutoff, their statistical output from Latent Gold, was then used to determine classification percentages. As can be seen from Table 13, Gene 2 column, the 2-gene model TGFB1 and CD69 correctly classified subjects suffering from NPG and POAG or normal subjects with a maximum classification rate of 94% and 92% accuracy, respectively. These results are depicted graphically in FIG. 3.

FIG. 3 also shows that a line can almost perfectly distinguish the two groups using the 2 gene model TGFB1 and CD69. This discrimination line is an example of the Index Function evaluated at a particular logit (log odds) value. Values above and to the left of the line are predicted to be in the normal, those below and to the right in the NPG and POAG population. This is a simplified version of the “Index function” as displayed in two dimensions, where the gene with positive coefficients (positive contributions) (CD69) is plotted along the horizontal axis, and the gene with negative coefficients (TGFB1) is plotted along the vertical axis. ‘Positive’ coefficients means that the higher the ΔC_{T} values for those genes (holding the other genes constant) increases the predicted logit, and thus the predicted probability of being in the diseased group.

The intercept (alpha) and slope (beta) of the discrimination line was computed according to the data shown in Table 14. A cutoff of 0.53681 was used to compute alpha (equals 0.147507 in logit units).

The following equation is given below the graph shown in FIG. 3:

NPG and POAG Discrimination Line: TGFB1=5.4355+0.3647*CD69.

Subjects below and to the right of this discrimination line have a predicted probability of being in the diseased groups higher than the cutoff probability of 0.53681.

The intercept C₀=5.43554 was computed by taking the SPSS regression value of 41.45 and subtracting the log-odds of the cutoff probability (0.147507). This quantity was then multiplied by −1/X where X is the coefficient for TGFB1 (−7.5986).

These data support that Gene Expression Profiles with sufficient precision and calibration as described herein (1) can determine subsets of individuals with a known biological condition, particularly individuals with ocular disease or individuals with conditions related to ocular disease; (2) may be used to monitor the response of patients to therapy; (3) may be used to assess the efficacy and safety of therapy; and (4) may be used to guide the medical management of a patient by adjusting therapy to bring one or more relevant Gene Expression Profiles closer to a target set of values, which may be normative values or other desired or achievable values.

Gene Expression Profiles are used for characterization and monitoring of treatment efficacy of individuals with ocular disease, or individuals with conditions related to ocular disease. Use of the algorithmic and statistical approaches discussed above to achieve such identification and to discriminate in such fashion is within the scope of various embodiments herein.

The references listed below are hereby incorporated herein by reference.

### REFERENCES

Magidson, J. GOLDMineR User's Guide (1998). Belmont, Mass.: Statistical Innovations Inc.
Vermunt J. K. and J. Magidson. Latent GOLD 4.0 User's Guide. (2005) Belmont, Mass.: Statistical Innovations Inc.
Vermunt J. K. and J. Magidson. Technical Guide for Latent GOLD 4.0: Basic and Advanced (2005)
Belmont, Mass.: Statistical Innovations Inc.
Vermunt J. K. and J. Magidson. Latent Class Cluster Analysis in (2002) J. A. Hagenaars and A. L. McCutcheon (eds.), Applied Latent Class Analysis, 89-106. Cambridge: Cambridge University Press.
Magidson, J. “Maximum Likelihood Assessment of Clinical Trials Based on an Ordered Categorical Response.” (1996) Drug Information Journal, Maple Glen, Pa.: Drug Information Association, Vol. 30, No. 1, pp 143-170.

TABLE 1A
Precision Profile ™ for Ocular Disease: Glaucoma

| Gene | | |
|---|---|---|
| Symbol | Alias(es) | Name |
| ADAM17 | CSVP, TACE, TNF-a converting | A Disintegrin and Metalloproteinase Domain 17 |
| | enzyme | |
| ANXA11 | CAP-50, ANX11, Annexin XI, 56 kDa | Annexin A11 |
| | autoantigen | |
| APAF1 | CED4, KIAA0413 | Apoptotic Protease Activating Factor 1 |
| APOE | Apo-E | Apolipoprotein E |
| BAD | BCL2L8, BBC2, BBC6, BCLX/BCL2 | BCL2 Agonist of Cell Death |
| | binding protein | |
| BAK1 | BAK, CDN1, BCL2L7, Cell death | BCL2-Antagonist/Killer 1 |
| | inhibitor 1 | |
| BAX | Apoptosis regulator Bax | BCL2-Associated X Protein |
| BCL2 | Apoptosis regulator Bcl-2 | B-Cell CLL/Lymphoma 2 |
| BCL2L1 | BCL-XL/S, BCL2L, BCLX, BCLXL, | BCL2-Like 1 (Long Form) |
| | BCLXS, Bcl-X | |
| BCL3 | BLC4, B-cell leukemia/lymphoma 3 | B-Cell CLL/Lymphoma 3 |
| BID | None | BH3-Interacting Death Domain Agonist |
| BIK | BIP1, BP4, NBK, BBC1 | BCL2-Interacting Killer |
| BIRC2 | API1, CIAP1, C-IAP, IAP1, MIHB, | Baculoviral IAP Repeat-Containing 2 |
| | MIHC | |
| BIRC3 | API2, C-IAP1, IAP2, MIHB; MIHC, | Baculoviral IAP Repeat-Containing 3 |
| | cIAP2 | |
| C1QA | C1QA1, Serum C1Q | Complement Component 1, Q Subcomponent, Alpha |
| | | Polypeptide |
| CASP1 | ICE, IL-1BC, IL1BC, IL1BCE, IL1B- | Caspase 1 |
| | convertase, P45 | |
| CASP3 | Yama, Apopain, CPP32, CPP32B, | Caspase 3 |
| | SCA-1 | |
| CASP9 | APAF3, MCH6, ICE-LAP6 | Caspase 9 |
| CAT | EC 1.11.1.6 | Catalase |
| CD19 | LEU12, B-lymphocyte antigen CD19 | CD19 Antigen |
| CD3Z | CD3-Zeta, CD3H, CD3Q, T3Z, TCRZ | CD3 Antigen, Zeta Polypeptide |
| CD4 | p55, T-cell antigen T4/leu3 | CD4 Antigen |
| CD44 | CD44R, IN, MC56, MDU2, MDU3, | CD44 Antigen |
| | MIC4, Pgp1, LHR | |
| CD68 | Macrosialin, GP110, SCARD1 | CD68 Antigen |
| CD69 | AIM, BL-AC/P26, EA1, GP32/28, | CD69 Antigen (p60, Early T-Cell Activation Antigen) |
| | Leu-23, MLR-3 | |
| CD8A | CD8, LEU2, MAL, p32, CD8 T-cell | CD8 Antigen, Alpha Polypeptide |
| | antigen LEU2 | |
| CRP | PTX1 | C-Reactive Protein, Pentraxin Related |
| CTGF | NOV2, IGFBP8, HCS24, CCN2, | Connective Tissue Growth Factor |
| | IGFBPR2 | |
| DIABLO | SMAC; SMAC3; DIABLO-S | diablo homolog (Drosophila) |
| ECE1 | ECE, ECE-1 | Endothelin Converting Enzyme 1 |
| EDN1 | ET1 | Endothelin 1 |
| FAIM3 | TOSO | Fas apoptotic inhibitory molecule 3 |
| FASLG | APT1LG1, CD178, CD95L, FASL, | Fas ligand (TNF superfamily, member 6) |
| | TNFSF6 | |
| FLT1 | FLT; VEGFR1 | fms-related tyrosine kinase 1 (vascular endothelial |
| | | growth factor/vascular permeability factor receptor) |
| GSR | GR, GRASE, GLUR, GRD1 | Glutathione Reductase |
| GSTA1 | GST2; GTH1; GSTA1-1; MGC131939 | glutathione S-transferase A1 |
| HIF1A | MOP1, ARNT Interacting Protein | Hypoxia-Inducible Factor 1, Alpha Subunit |
| HLA-DRB1 | HLA class II histocompatibility | Major Histocompatibility Complex, Class II, DR Beta 1 |
| | antigen, DR-1 beta chain | |
| HSPA1A | HSP-70, HSP70-1 | Heat Shock Protein 1A, 70 kD |
| IFNG | IFG, IFI, IFN-g | Interferon, Gamma |
| IL10 | CSIF, IL-10, TGIF, Cytokine synthesis | Interleukin 10 |
| | inhibitory factor | |
| IL1RN | ICIL-1RA, IL1F3, IL-1RA, IRAP, IL- | Interleukin 1 Receptor Antagonist |
| | 1RN, IL1RA | |
| IL2 | TCGF | Interleukin 2 |
| IL2RA | IL2R, P55, TCGFR, CD25, TAC | Interleukin 2 Receptor, Alpha |
| | antigen | |
| IL6 | Interferon beta 2, IFNB2, BSF2, HSF | Interleukin 6 |
| IL8 | CXCL8, SCYB8, MDNCF | Interleukin 8 |
| JUN | CJUN, Proto-oncogene c-Jun, AP-1, | V-jun Avian Sarcoma Virus 17 Oncogene Homolog |
| | AP1 | |
| LTA | TNFSF1, Tumor necrosis factor beta | Lymphotoxin, Alpha |
| | (formerly), TNFB | |
| MADD | DENN, IG20, Insulinoma- | MAP-Kinase Activating Death Domain |
| | glucagonoma protein 20 | |
| MAP3K1 | MAPKKK1, MEKK1, MEKK, | Mitogen-Activated Protein Kinase Kinase Kinase 1 |
| | MAP/ERK kinase kinase 1 | |
| MAP3K14 | NF-kB Inducing Kinase, NIK, HSNIK, | Mitogen-Activated Protein Kinase Kinase Kinase 14 |
| | FTDCR1B, HS | |
| MAPK1 | ERK2, ERK, ERT1, MAPK2, PRKM1, | Mitogen-Activated Protein Kinase 1 |
| | p38, p40, p41 | |
| MAPK14 | CSBP, CSBP1, p38, Mxi2, PRKM14, | Mitogen-Activated Protein Kinase 14 |
| | PRKM15 | |
| MAPK8 | JNK1, JNK, SAPK1, PRKM8, | Mitogen-Activated Protein Kinase 8 |
| | JNK1A2, JNK21B1/2 | |
| MMP1 | Collagenase, CLG, CLGN, Fibroblast | Matrix Metalloproteinase 1 |
| | collagenase | |
| MMP12 | Macrophage elastase, HME, MME | Matrix Metalloproteinase 12 |
| MMP13 | Collagenase 3, CLG3 | Matrix Metalloproteinase 13 |
| MMP15 | MT2-MMP, MMP-15, SMCP-2, | Matrix Metalloproteinase 15 (Membrane-Inserted) |
| | MT2MMP, MTMMP2 | |
| MMP19 | MMP18 (formerly), RASI-1, RASI | Matrix Metalloproteinase 19 |
| MMP2 | Gelatinase, CLG4A, CLG4, TBE-1, | Matrix Metalloproteinase 2 |
| | Gelatinase A | |
| MMP3 | Stromelysin, STMY1, STMY, SL-1, | Matrix Metalloproteinase 3 |
| | STR1, Transin-1 | |
| MMP8 | Neutrophil collagenase, CLG1, HNCl, | Matrix Metalloproteinase 8 |
| | PMNL-CL | |
| MMP9 | Gelatinase B, CLG4B, GELB, | Matrix Metalloproteinase 9 |
| | Macrophage gelatinase | |
| NFKB1 | KBF1, EBP-1, NFKB p50 | Nuclear Factor of Kappa Light Polypeptide Gene |
| | | Enhancer in B-Cells 1 (p105) |
| NFKBIB | TRIP9, IKBB, Thyroid hormone | Nuclear Factor of Kappa Light Polypeptide Gene |
| | receptor interactor 9 | Enhancer in B-Cells Inhibitor, Beta |
| NOS1 | NOS, N-NOS, NNOS, Neuronal NOS, | Nitric Oxide Synthase 1 (Neuronal) |
| | Constitutive NOS | |
| NOS2A | iNOS, NOS2 | Nitric Oxide Synthase 2A (Inducible) |
| NOS3 | eNOS, cNOS, ECNOS | Nitric Oxide Synthase 3 (Endothelial) |
| PDCD8 | AIF, Apoptosis-Inducing Factor | Programmed Cell Death 8 |
| PLAU | UPA, URK, Plasminogen activator | Plasminogen Activator, Urokinase |
| | (urinary) | |
| PPARA | PPAR, HPPAR, NR1C1 | Peroxisome Proliferator Activated Receptor, Alpha |
| PPARG | HUMPPARG, NR1C3, PPAR-g, | Peroxisome Proliferator Activated Receptor, Gamma |
| | PPARG3, PPARG2, PPARG1 | |
| PTGS2 | COX2, COX-2, PGG/HS, PGHS-2, | Prostaglandin-Endoperoxide Synthase 2 |
| | PHS-2, hCox-2 | |
| SAA1 | SAA; PIG4; TP53I4; MGC111216 | serum amyloid A1 |
| SERPINA3 | AACT, ACT, Alpha-1-Anti- | Serine (or Cysteine) Proteinase Inhibitor, Clade A, |
| | chymotrypsin | Member 3 |
| SERPINB2 | PAI, PAI-2, PAI2, PLANH2, | Serine (or Cysteine) Proteinase Inhibitor, Clade B |
| | Urokinase inhibitor | (Ovalbumin), Member 2 |
| SOD2 | IPO-B, MnSOD, Indophenoloxidase B | Superoxide Dismutase 2 (Mitochondrial) |
| TGFA | ETGF, TGF-alpha, EGF-like TGF, | Transforming Growth Factor, Alpha |
| | TGF type 1 | |
| TGFB1 | DPD1, CED, HGNC: 2997, TGF-beta, | Transforming Growth Factor, Beta 1 |
| | TGFB, TGF-b | |
| TGFB3 | TGF-b3 | Transforming Growth Factor, Beta 3 |
| TIMP1 | TIMP, Erythroid potentiating activity, | Tissue Inhibitor of Matrix Metalloproteinase 1 |
| | CLGI, EPA, EPO, HCI | |
| TIMP3 | SFD, HSMRK222, K222TA2 | Tissue Inhibitor of Matrix Metalloproteinase 3 |
| TNF | TNF-alpha, TNFa, cachectin, DIF, | Tumor Necrosis Factor, Member 2 |
| | TNFA, TNFSF2 | |
| TNFRSF11A | RANK, Activator of NF-kB, ODFR, | Tumor Necrosis Factor Receptor Superfamily, Member |
| | PDB2 | 11A |
| TNFRSF13B | TACI, Transmembrane Activator & | Tumor Necrosis Factor Receptor Superfamily, Member |
| | CAML Interactor | 13B |
| TNFRSF1A | FPF, TNF-R, TNF-R1, TNFAR, | Tumor Necrosis Factor Receptor Superfamily, Member |
| | TNFR1, TNFR60, p55, p55-R | 1A |
| TNFRSF1B | TNFR2, p75, CD120b | Tumor Necrosis Factor Receptor Superfamily, Member |
| | | 1B |
| TNFRSF25 | TNFRSF12 (formerly), LARD, | Tumor Necrosis Factor Receptor Superfamily, Member |
| | TRAMP, WSL-1, TR3, DR3 | 25 |
| TNFSF12 | TWEAK, APO3L, DR3LG | Tumor Necrosis Factor (Ligand) Superfamily, Member |
| | | 12 |
| TP53 | p53, TRP53 | Tumor Protein 53 (Li-Fraumeni Syndrome) |
| TRADD | Tumor necrosis factor receptor-1- | TNFRSF1A-Associated Via Death Domain |
| | associated protein | |
| TRAF1 | EBI6, MGC: 10353, Epstein-barr virus- | TNF Receptor-Associated Factor 1 |
| | induced mRNA 6 | |
| TRAF2 | TNF-receptor-associated protein, | TNF Receptor-Associated Factor 2 |
| | MGC: 45012, TRAP3 | |
| TRAF3 | CD40BP, LAP1, CAP1, CRAF1, | TNF Receptor-Associated Factor 3 |
| | LMP1 | |
| TXNRD1 | TXNR, TR1 | Thioredoxin Reductase 1 |
| VDAC1 | PORIN, PORIN-31-HL, Plasmalemmal | Voltage-Dependent Anion Channel 1 |
| | porin | |

TABLE 1B
Precision Profile ™ for Ocular Disease: Age Related Macular Degeneration (AMD)

| Gene | | | Accession |
|---|---|---|---|
| Symbol | Alias(es) | Name | Number |
| ADAM17 | CSVP, TACE, TNF-a converting | A Disintegrin and Metalloproteinase | NM_003183 |
| | enzyme | Domain 17 | |
| ADAMTS1 | METH1, C3-C5, KIAA1346 | A Disintegrin-Like and | NM_006988 |
| | | Metalloproteinase (Reprolysin Type) | |
| | | with Thrombospondin Type 1 Motif, 1 | |
| ALOX5 | RP11-67C2.3, 5-LO, 5LPG, LOG5 | Arachidonate 5-Lipoxygenase | NM_000698 |
| APAF1 | CED4, KIAA0413 | Apoptotic Protease Activating Factor 1 | NM_013229 |
| APOE | Apo-E | Apolipoprotein E | NM_000041 |
| BAD | BCL2L8, BBC2, BBC6, | BCL2 Agonist of Cell Death | NM_004322 |
| | BCLX/BCL2 binding protein | | |
| BAK1 | BAK, CDN1, BCL2L7, Cell death | BCL2-Antagonist/Killer 1 | NM_001188 |
| | inhibitor 1 | | |
| BAX | Apoptosis regulator Bax | BCL2-Associated X Protein | NM_138761 |
| BCL2 | Apoptosis regulator Bcl-2 | B-Cell CLL/Lymphoma 2 | NM_000633 |
| BCL2L1 | BCL-XL/S, BCL2L, BCLX, | BCL2-Like 1 (Long Form) | NM_001191 |
| | BCLXL, BCLXS, Bcl-X | | |
| BCL3 | BLC4, B-cell leukemia/lymphoma 3 | B-Cell CLL/Lymphoma 3 | NM_005178 |
| BID | None | BH3-Interacting Death Domain | NM_197966 |
| | | Agonist | |
| BIK | BIP1, BP4, NBK, BBC1 | BCL2-Interacting Killer | NM_001197 |
| BIRC2 | API1, CIAP1, C-IAP, IAP1, MIHB, | Baculoviral IAP Repeat-Containing 2 | NM_001166 |
| | MIHC | | |
| BIRC3 | API2, C-IAP1, IAP2, MIHB; | Baculoviral IAP Repeat-Containing 3 | NM_001165 |
| | MIHC, cIAP2 | | |
| BSG | EMMPRIN, 5F7, CD147, OK, M6, | Basignin (OK Blood Group) | NM_001728 |
| | TCSF | | |
| C1QA | C1QA1, Serum C1Q | Complement Component 1, Q | NM_015991 |
| | | Subcomponent, Alpha Polypeptide | |
| C1QB | None | Complement Component 1, Q | NM_000491 |
| | | Subcomponent, Beta Polypeptide | |
| CASP1 | ICE, IL-1BC, IL1BC, IL1BCE, | Caspase 1 | NM_033292 |
| | IL1B-convertase, P45 | | |
| CASP3 | Yama, Apopain, CPP32, CPP32B, | Caspase 3 | NM_004346 |
| | SCA-1 | | |
| CASP9 | APAF3, MCH6, ICE-LAP6 | Caspase 9 | NM_001229 |
| CAT | EC 1.11.1.6 | Catalase | NM_001752 |
| CCL2 | SCYA2, MCP1, HC11, MCAF, | Chemokine (C-C Motif) Ligand 2 | NM_002982 |
| | MGC9434, SMC-CF | | |
| CCL3 | SCYA3, LD78-Alpha, MIP1A, | Chemokine (C-C Motif) Ligand 3 | NM_002983 |
| | SIS-beta, G0S19-1 | | |
| CCL5 | SCYA5, D17S136E, RANTES, | Chemokine (C-C Motif) Ligand 5 | NM_002985 |
| | TCP228 | | |
| CCL7 | MCP-3, NC28, FIC, MARC | Chemokine (C-C Motif) Ligand 7 | NM_006273 |
| | SCYA6, SCYA7 | | |
| CCL8 | MCP-2, MCP2, HC14, SCYA8, | Chemokine (C-C Motif) Ligand 8 | NM_005623 |
| | SCYA10 | | |
| CCR1 | CC-CKR-1, CMKR1, MIP1aR, | Chemokine (C-C motif) Receptor 1 | NM_001295 |
| | RANTES-R, SCYAR1 | | |
| CCR3 | CC-CKR-3, CMKBR3, CKR3, | Chemokine (C-C motif) Receptor 3 | NM_001837 |
| | Eotaxin receptor | | |
| CCR5 | CKR-5, CKR5, chemr13, CC-CKR- | Chemokine (C-C motif) Receptor 5 | NM_000579 |
| | 5, CMKBR5 | | |
| CD34 | Hematopoietic progenitor cell | CD34 Antigen | NM_001773 |
| | antigen, HPCA1 | | |
| CD4 | p55, T-cell antigen T4/leu3 | CD4 Antigen | NM_000616 |
| CD44 | CD44R, IN, MC56, MDU2, | CD44 Antigen | NM_000610 |
| | MDU3, MIC4, Pgp1, LHR | | |
| CD48 | BCM1, BLAST, Lymphocyte | CD48 Antigen | NM_001778 |
| | antigen, MEM-102, BLAST1 | | |
| CD80 | CD28LG, CD28LG1, LAB7 | CD80 molecule | NM_005191 |
| CD8A | CD8, LEU2, MAL, p32, CD8 T- | CD8 Antigen, Alpha Polypeptide | NM_001768 |
| | cell antigen LEU2 | | |
| CRP | PTX1 | C-Reactive Protein, Pentraxin Related | NM_000567 |
| CTGF | NOV2, IGFBP8, HCS24, CCN2, | Connective Tissue Growth Factor | NM_001901 |
| | IGFBPR2 | | |
| CTNNA1 | Cadherin-associated protein, | Catenin, Alpha 1 | NM_001903 |
| | CAP102 | | |
| CTSB | APPS, CPSB, APP secretase | Cathepsin B | NM_001908 |
| CXCL1 | GRO1; GROa; MGSA; NAP-3; | chemokine (C—X—C motif) ligand 1 | NM_001511 |
| | SCYB1; MGSA-a; MGSA alpha | (melanoma growth stimulating activity, | |
| | | alpha) | |
| CXCL2 | GRO2; GROb; MIP2; MIP2A; | chemokine (C—X—C motif) ligand 2 | NM_002089 |
| | SCYB2; MGSA-b; MIP-2a; CINC- | | |
| | 2a; MGSA beta | | |
| CXCR3 | GPR9, CD183, CKR-L2, IP10-R, | Chemokine (C—X—C Motif) Receptor 3 | NM_001504 |
| | Mig-R, MigR, IP10 | | |
| DIABLO | SMAC; SMAC3; DIABLO-S | diablo homolog (Drosophila) | NM_019887 |
| ECE1 | ECE, ECE-1 | Endothelin Converting Enzyme 1 | NM_001397 |
| ELA2 | Medullasin, NE, SERP1, PMN | Elastase 2, Neutrophil | NM_001972 |
| | elastase | | |
| FADD | MORT1, MGC8528, Mediator of | Fas (TNFRSF6)-Associated Via Death | NM_003824 |
| | receptor-induced toxicity | Domain | |
| FASLG | APT1LG1, CD178, CD95L, FASL, | Fas ligand (TNF superfamily, member | NM_000639 |
| | TNFSF6 | 6) | |
| FGF2 | BFGF, FGFB, HBGF-2, HBGH-2, | Fibroblast Growth Factor 2 (Basic) | NM_002006 |
| | Prostatropin | | |
| FLT1 | VEGFR1, FRT, FLT | FMS-Related Tyrosine Kinase 1 | NM_002019 |
| FN1 | CIG, FN, LETS, LETS FNZ, FINC | Fibronectin 1 | NM_002026 |
| HIF1A | MOP1, ARNT Interacting Protein | Hypoxia-Inducible Factor 1, Alpha | NM_001530 |
| | | Subunit | |
| HLA-DRB1 | HLA class II histocompatibility | Major Histocompatibility Complex, | NM_002124 |
| | antigen, DR-1 beta chain | Class II, DR Beta 1 | |
| ICAM1 | CD54, BB2, Human rhinovirus | Intercellular Adhesion Molecule 1 | NM_000201 |
| | receptor | | |
| IFNA2_8_10 | LeIF-A; LeiF-B; LelF-C | Interferon, Alpha 2; Interferon, Alpha | NM_000605 |
| | | 8; Interferon, Alpha 10 | |
| IFNG | IFG, IFI, IFN-g | Interferon, Gamma | NM_000619 |
| IL1RN | ICIL-1RA, IL1F3, IL-1RA, IRAP, | Interleukin 1 Receptor Antagonist | NM_173843 |
| | IL-1RN, IL1RA | | |
| IL2 | TCGF | Interleukin 2 | NM_000586 |
| IL6 | Interferon beta 2, IFNB2, BSF2, | Interleukin 6 | NM_000600 |
| | HSF | | |
| IL8 | CXCL8, SCYB8, MDNCF | Interleukin 8 | NM_000584 |
| MMP1 | Collagenase, CLG, CLGN, | Matrix Metalloproteinase 1 | NM_002421 |
| | Fibroblast collagenase | | |
| MMP12 | Macrophage elastase, HME, MME | Matrix Metalloproteinase 12 | NM_002426 |
| MMP19 | MMP18 (formerly), RASI-1, RASI | Matrix Metalloproteinase 19 | NM_002429 |
| MMP2 | Gelatinase, CLG4A, CLG4, TBE-1, | Matrix Metalloproteinase 2 | NM_004530 |
| | Gelatinase A | | |
| MMP3 | Stromelysin, STMY1, STMY, SL- | Matrix Metalloproteinase 3 | NM_002422 |
| | 1, STR1, Transin-1 | | |
| MMP9 | Gelatinase B, CLG4B, GELB, | Matrix Metalloproteinase 9 | NM_004994 |
| | Macrophage gelatinase | | |
| NFKB1 | KBF1, EBP-1, NFKB p50 | Nuclear Factor of Kappa Light | NM_003998 |
| | | Polypeptide Gene Enhancer in B-Cells | |
| | | 1 (p105) | |
| NOS1 | NOS, N-NOS, NNOS, Neuronal | Nitric Oxide Synthase 1 (Neuronal) | NM_000620 |
| | NOS, Constitutive NOS | | |
| NOS2A | iNOS, NOS2 | Nitric Oxide Synthase 2A (Inducible) | NM_000625 |
| NRP1 | NRP, VEGF165R | Neuropilin 1 | NM_003873 |
| PITRM1 | MP1, hMP1, KIAA1104 | Pitrilysin Metalloproteinase 1 | NM_014889 |
| PLAT | TPA, T-PA, Alteplase, Reteplase | Plasminogen Activator, Tissue | NM_000930 |
| PLAU | UPA, URK, Plasminogen activator | Plasminogen Activator, Urokinase | NM_002658 |
| | (urinary) | | |
| PPARA | PPAR, HPPAR, NR1C1 | Peroxisome Proliferator Activated | NM_001001930 |
| | | Receptor, Alpha | |
| PPARG | HUMPPARG, NR1C3, PPAR-g, | Peroxisome Proliferator Activated | NM_138712 |
| | PPARG3, PPARG2, PPARG1 | Receptor, Gamma | |
| PTGS1 | COX1, COX-1, PGG/HS, PGHS1, | Prostaglandin-Endoperoxide Synthase 1 | NM_000962 |
| | PTGHS | | |
| PTGS2 | COX2, COX-2, PGG/HS, PGHS-2, | Prostaglandin-Endoperoxide Synthase 2 | NM_000963 |
| | PHS-2, hCox-2 | | |
| SAA1 | SAA; PIG4; TP53I4; MGC111216 | serum amyloid A1 | NM_199161 |
| SELE | ELAM, CD62E, ELAM1, ESEL, | Selectin E | NM_000450 |
| | LECAM2 | | |
| SERPINA1 | Alpha 1 Anti-proteinase, AAT, PI1, | Serine (or Cysteine) Proteinase | NM_000295 |
| | PI, A1AT | Inhibitor, Clade A, Member 1 | |
| SERPINA3 | AACT, ACT, Alpha-1-Anti- | Serine (or Cysteine) Proteinase | NM_001185 |
| | chymotrypsin | Inhibitor, Clade A, Member 3 | |
| SERPINB2 | PAI, PAI-2, PAI2, PLANH2, | Serine (or Cysteine) Proteinase | NM_002575 |
| | Urokinase inhibitor | Inhibitor, Clade B (Ovalbumin), | |
| | | Member 2 | |
| SERPINE1 | PAI1, Plasminogen activator | Serine (or Cysteine) Proteinase | NM_000602 |
| | inhibitor type 1, PAIE, PLANH1 | Inhibitor, Clade E (Ovalbumin), | |
| | | Member 1 | |
| SERPING1 | C-1 esterase inhibitor, C1NH, C1- | Serine (or Cysteine) Proteinase | NM_000062 |
| | INH, C1I, HAE1, HAE2 | Inhibitor, Clade G (C1 Inhibitor), | |
| | | Member 1 (Angioedema, Hereditary) | |
| SOD2 | IPO-B, MnSOD, | Superoxide Dismutase 2 | NM_000636 |
| | Indophenoloxidase B | (Mitochondrial) | |
| TGFA | ETGF, TGF-alpha, EGF-like TGF, | Transforming Growth Factor, Alpha | NM_003236 |
| | TGF type 1 | | |
| TGFB1 | DPD1, CED, HGNC: 2997, TGF- | Transforming Growth Factor, Beta 1 | NM_000660 |
| | beta, TGFB, TGF-b | | |
| TGFB3 | TGF-b3 | Transforming Growth Factor, Beta 3 | NM_003239 |
| TIMP1 | TIMP, Erythroid potentiating | Tissue Inhibitor of Matrix | NM_003254 |
| | activity, CLGI, EPA, EPO, HCI | Metalloproteinase 1 | |
| TIMP3 | SFD, HSMRK222, K222TA2 | Tissue Inhibitor of Matrix | NM_000362 |
| | | Metalloproteinase 3 | |
| TNF | TNF-alpha, TNFa, cachectin, DIF, | Tumor Necrosis Factor, Member 2 | NM_000594 |
| | TNFA, TNFSF2 | | |
| TNFRSF11A | RANK, Activator of NF-kB, | Tumor Necrosis Factor Receptor | NM_003839 |
| | ODFR, PDB2 | Superfamily, Member 11A | |
| TNFRSF1A | FPF, TNF-R, TNF-R1, TNFAR, | Tumor Necrosis Factor Receptor | NM_001065 |
| | TNFR1, TNFR60, p55, p55-R | Superfamily, Member 1A | |
| TNFRSF1B | TNFR2, p75, CD120b | Tumor Necrosis Factor Receptor | NM_001066 |
| | | Superfamily, Member 1B | |
| TNFRSF25 | TNFRSF12 (formerly), LARD, | Tumor Necrosis Factor Receptor | NM_148965 |
| | TRAMP, WSL-1, TR3, DR3 | Superfamily, Member 25 | |
| VCAM1 | L1CAM, CD106, INCAM-100 | Vascular Cell Adhesion Molecule 1 | NM_001078 |
| VEGF | VPF, VEGF-A, VEGFA, | vascular endothelial growth factor A | NM_003376 |
| | Vasculotropin | | |

TABLE 2
Precision Profile ™ for Inflammatory Response

| Gene | | Gene Accession |
|---|---|---|
| Symbol | Gene Name | Number |
| ADAM17 | a disintegrin and metalloproteinase domain 17 (tumor necrosis factor, | NM_003183 |
| | alpha, converting enzyme) | |
| ALOX5 | arachidonate 5-lipoxygenase | NM_000698 |
| ANXA11 | annexin A11 | NM_001157 |
| APAF1 | apoptotic Protease Activating Factor 1 | NM_013229 |
| BAX | BCL2-associated X protein | NM_138761 |
| C1QA | complement component 1, q subcomponent, alpha polypeptide | NM_015991 |
| CASP1 | caspase 1, apoptosis-related cysteine peptidase (interleukin 1, beta, | NM_033292 |
| | convertase) | |
| CASP3 | caspase 3, apoptosis-related cysteine peptidase | NM_004346 |
| CCL2 | chemokine (C-C motif) ligand 2 | NM_002982 |
| CCL3 | chemokine (C-C motif) ligand 3 | NM_002983 |
| CCL5 | chemokine (C-C motif) ligand 5 | NM_002985 |
| CCR3 | chemokine (C-C motif) receptor 3 | NM_001837 |
| CCR5 | chemokine (C-C motif) receptor 5 | NM_000579 |
| CD14 | CD14 antigen | NM_000591 |
| CD19 | CD19 Antigen | NM_001770 |
| CD4 | CD4 antigen (p55) | NM_000616 |
| CD86 | CD86 antigen (CD28 antigen ligand 2, B7-2 antigen) | NM_006889 |
| CD8A | CD8 antigen, alpha polypeptide | NM_001768 |
| CRP | C-reactive protein, pentraxin-related | NM_000567 |
| CSF2 | colony stimulating factor 2 (granulocyte-macrophage) | NM_000758 |
| CSF3 | colony stimulating factor 3 (granulocytes) | NM_000759 |
| CTLA4 | cytotoxic T-lymphocyte-associated protein 4 | NM_005214 |
| CXCL1 | chemokine (C—X—C motif) ligand 1 (melanoma growth stimulating | NM_001511 |
| | activity, alpha) | |
| CXCL10 | chemokine (C—X—C moif) ligand 10 | NM_001565 |
| CXCL3 | chemokine (C—X—C motif) ligand 3 | NM_002090 |
| CXCL5 | chemokine (C—X—C motif) ligand 5 | NM_002994 |
| CXCR3 | chemokine (C—X—C motif) receptor 3 | NM_001504 |
| DPP4 | Dipeptidylpeptidase 4 | NM_001935 |
| EGR1 | early growth response-1 | NM_001964 |
| ELA2 | elastase 2, neutrophil | NM_001972 |
| FAIM3 | Fas apoptotic inhibitory molecule 3 | NM_005449 |
| FASLG | Fas ligand (TNF superfamily, member 6) | NM_000639 |
| GCLC | glutamate-cysteine ligase, catalytic subunit | NM_001498 |
| GZMB | granzyme B (granzyme 2, cytotoxic T-lymphocyte-associated serine | NM_004131 |
| | esterase 1) | |
| HLA-DRA | major histocompatibility complex, class II, DR alpha | NM_019111 |
| HMGB1 | high-mobility group box 1 | NM_002128 |
| HMOX1 | heme oxygenase (decycling) 1 | NM_002133 |
| HSPA1A | heat shock protein 70 | NM_005345 |
| ICAM1 | Intercellular adhesion molecule 1 | NM_000201 |
| ICOS | inducible T-cell co-stimulator | NM_012092 |
| IFI16 | interferon inducible protein 16, gamma | NM_005531 |
| IFNG | interferon gamma | NM_000619 |
| IL10 | interleukin 10 | NM_000572 |
| IL12B | interleukin 12 p40 | NM_002187 |
| IL13 | interleukin 13 | NM_002188 |
| IL15 | Interleukin 15 | NM_000585 |
| IRF1 | interferon regulatory factor 1 | NM_002198 |
| IL18 | interleukin 18 | NM_001562 |
| IL18BP | IL-18 Binding Protein | NM_005699 |
| IL1A | interleukin 1, alpha | NM_000575 |
| IL1B | interleukin 1, beta | NM_000576 |
| IL1R1 | interleukin 1 receptor, type I | NM_000877 |
| IL1RN | interleukin 1 receptor antagonist | NM_173843 |
| IL2 | interleukin 2 | NM_000586 |
| IL23A | interleukin 23, alpha subunit p19 | NM_016584 |
| IL32 | interleukin 32 | NM_001012631 |
| IL4 | interleukin 4 | NM_000589 |
| IL5 | interleukin 5 (colony-stimulating factor, eosinophil) | NM_000879 |
| IL6 | interleukin 6 (interferon, beta 2) | NM_000600 |
| IL8 | interleukin 8 | NM_000584 |
| LTA | lymphotoxin alpha (TNF superfamily, member 1) | NM_000595 |
| MAP3K1 | mitogen-activated protein kinase kinase kinase 1 | XM_042066 |
| MAPK14 | mitogen-activated protein kinase 14 | NM_001315 |
| MHC2TA | class II, major histocompatibility complex, transactivator | NM_000246 |
| MIF | macrophage migration inhibitory factor (glycosylation-inhibiting factor) | NM_002415 |
| MMP12 | matrix metallopeptidase 12 (macrophage elastase) | NM_002426 |
| MMP8 | matrix metallopeptidase 8 (neutrophil collagenase) | NM_002424 |
| MMP9 | matrix metallopeptidase 9 (gelatinase B, 92 kDa gelatinase, 92 kDa type | NM_004994 |
| | IV collagenase) | |
| MNDA | myeloid cell nuclear differentiation antigen | NM_002432 |
| MPO | myeloperoxidase | NM_000250 |
| MYC | v-myc myelocytomatosis viral oncogene homolog (avian) | NM_002467 |
| NFKB1 | nuclear factor of kappa light polypeptide gene enhancer in B-cells 1 | NM_003998 |
| | (p105) | |
| NOS2A | nitric oxide synthase 2A (inducible, hepatocytes) | NM_000625 |
| PLA2G2A | phospholipase A2, group IIA (platelets, synovial fluid) | NM_000300 |
| PLA2G7 | phospholipase A2, group VII (platelet-activating factor acetylhydrolase, | NM_005084 |
| | plasma) | |
| PLAU | plasminogen activator, urokinase | NM_002658 |
| PLAUR | plasminogen activator, urokinase receptor | NM_002659 |
| PRTN3 | proteinase 3 (serine proteinase, neutrophil, Wegener granulomatosis | NM_002777 |
| | autoantigen) | |
| PTGS2 | prostaglandin-endoperoxide synthase 2 (prostaglandin G/H synthase and | NM_000963 |
| | cyclooxygenase) | |
| PTPRC | protein tyrosine phosphatase, receptor type, C | NM_002838 |
| PTX3 | pentraxin-related gene, rapidly induced by IL-1 beta | NM_002852 |
| SERPINA1 | serine (or cysteine) proteinase inhibitor, clade A (alpha-1 antiproteinase, | NM_000295 |
| | antitrypsin), member 1 | |
| SERPINE1 | serpin peptidase inhibitor, clade E (nexin, plasminogen activator | NM_000602 |
| | inhibitor type 1), member 1 | |
| SSI-3 | suppressor of cytokine signaling 3 | NM_003955 |
| TGFB1 | transforming growth factor, beta 1 (Camurati-Engelmann disease) | NM_000660 |
| TIMP1 | tissue inhibitor of metalloproteinase 1 | NM_003254 |
| TLR2 | toll-like receptor 2 | NM_003264 |
| TLR4 | toll-like receptor 4 | NM_003266 |
| TNF | tumor necrosis factor (TNF superfamily, member 2) | NM_000594 |
| TNFRSF13B | tumor necrosis factor receptor superfamily, member 13B | NM_012452 |
| TNFRSF17 | tumor necrosis factor receptor superfamily, member 17 | NM_001192 |
| TNFRSF1A | tumor necrosis factor receptor superfamily, member 1A | NM_001065 |
| TNFSF13B | Tumor necrosis factor (ligand) superfamily, member 13b | NM_006573 |
| TNFSF5 | CD40 ligand (TNF superfamily, member 5, hyper-IgM syndrome) | NM_000074 |
| TXNRD1 | thioredoxin reductase | NM_003330 |
| VEGF | vascular endothelial growth factor | NM_003376 |

TABLE 3
NPG Study:
Ranking of genes from Table 1A from most to least significant:
1-Way ANOVA Approach

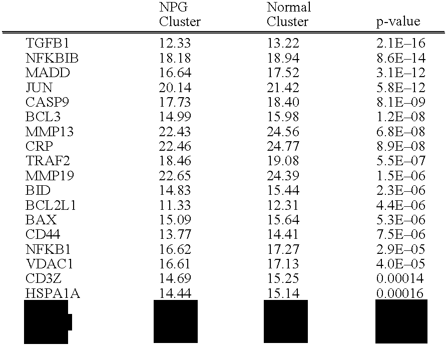

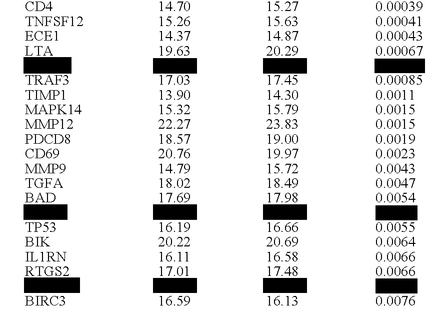

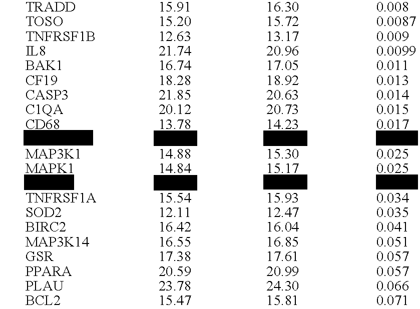

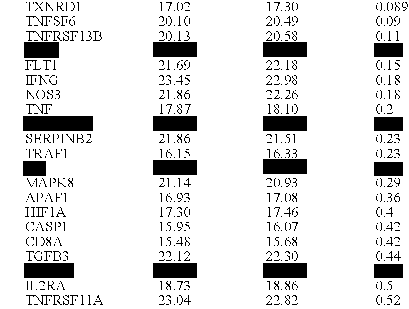

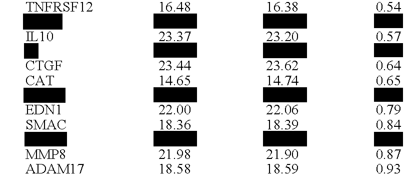

TABLE 4
NPG Study:
Ranking of genes based on Table 1A from most to least significant:
Stepwise logistic regression

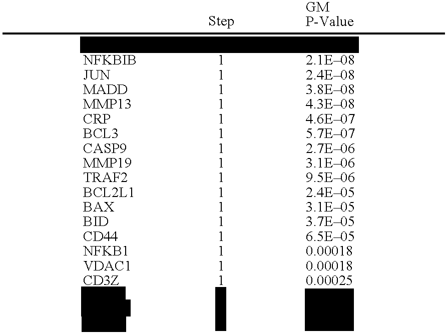

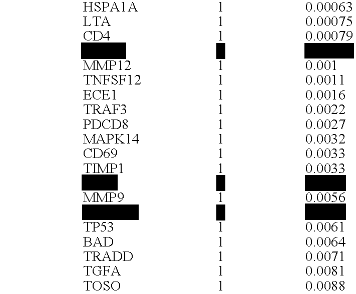

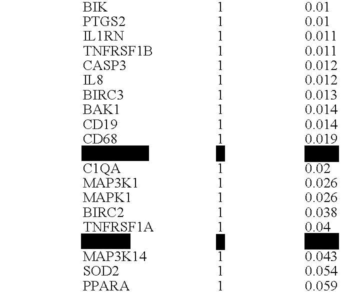

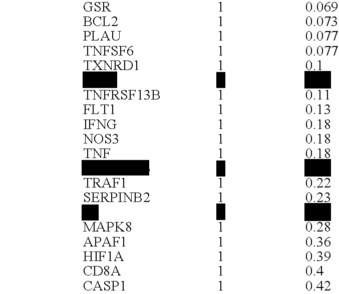

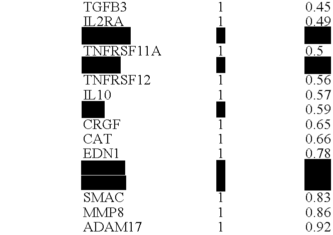

TABLE 5
1 and 2-gene NPG Models using TGFB1 as the initial gene

| | 1 Gene | | | 2 Gene | |
|---|---|---|---|---|---|
| | % NPG | % Normal | | % NPG | % Normal |
| Maximum = | 100% | 92% | Maximum = | 100% | 92% |
| TGFB1 | | | TGFB1 | | |
| | | | SERPINB2 | | |

TABLE 6
Data for NPG Discrimination Line

| | group | Class1 | | |
|---|---|---|---|---|
| | Intercept | | | Alpha |
| cutoff = | NPG | 34.3695 | 68.739 | 7.479153 |
| 0.3289 | normal | −34.3695 | | |
| −0.7131644 | | | | |
| | Predictors | Class1 | | |
| | TGFB1 | −9.2861 | | |
| | | | | Beta |
| | SERPINB2 | 2.2724 | | 0.24471 |

TABLE 7
POAG Study:
Ranking of genes based on Table 1A from most to least significant:
1-Way ANOVA Approach

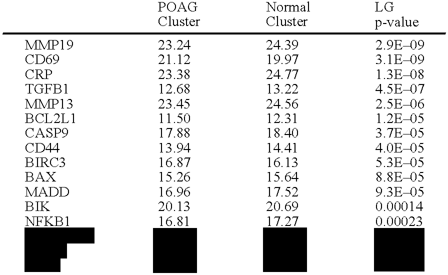

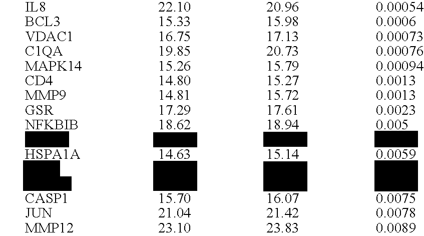

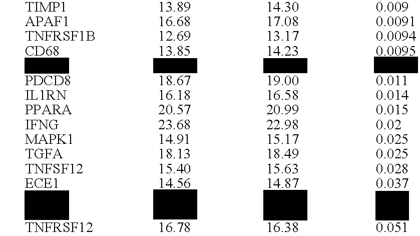

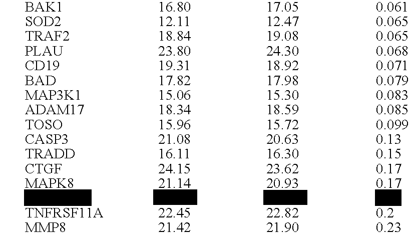

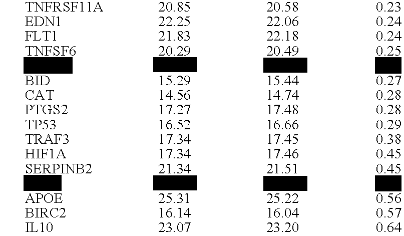

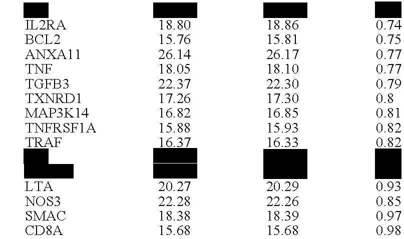

TABLE 8
POAG Study:
Ranking of genes based on Table 1A from most to least significant:
Stepwise logistic regression

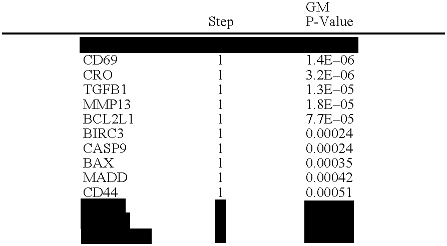

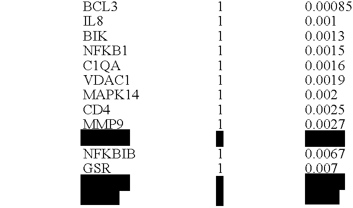

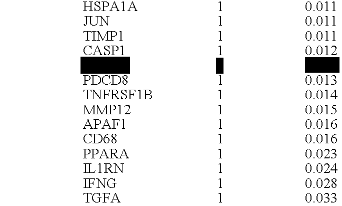

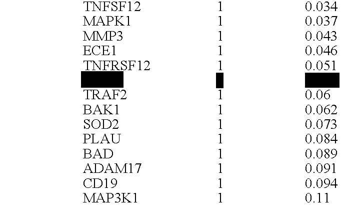

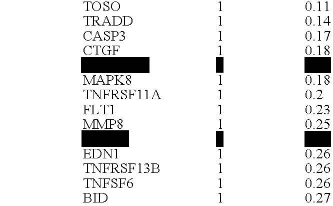

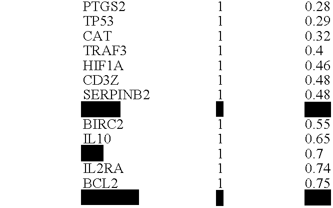

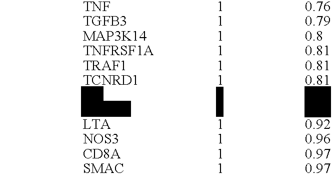

TABLE 9
1 and 2-gene POAG Models using MMP19 as the initial gene

| | 1 Gene | | | 2 Gene | % |
|---|---|---|---|---|---|
| | % POAG | % Normal | | % POAG | Normal |
| Maximum = | 77% | 92% | Maximum = | 88% | 96% |
| Adjusted = | 82% | 83% | Adjusted = | 94% | 92% |
| MMP19 | | | MMP19 | | |
| | | | CD69 | | |

TABLE 10
Data for POAG Discrimination Line

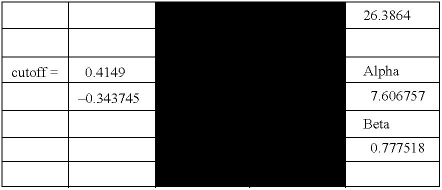

TABLE 11
Combined NPG and POAG Study:
Ranking of genes based on Table 1A from most to least significant:
1-Way ANOVA Approach

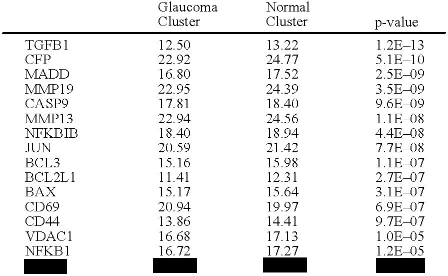

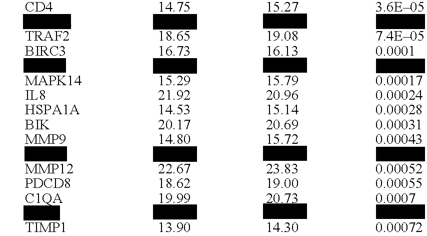

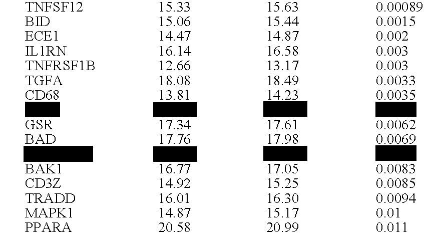

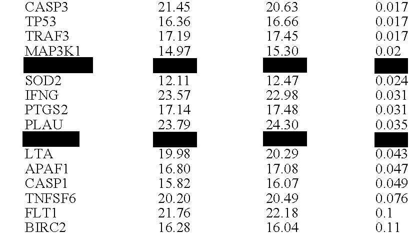

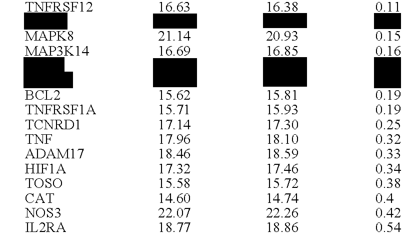

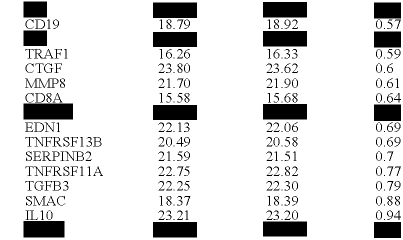

TABLE 12
Combine NPG and POAG Study:
Ranking of genes based on Table 1A from most to least significant:
Stepwise logistic regession

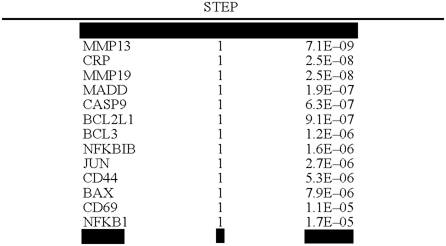

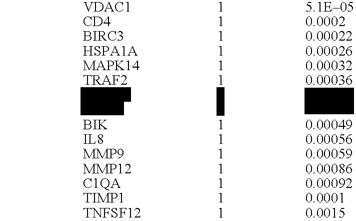

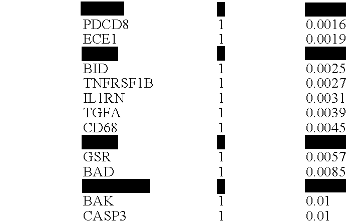

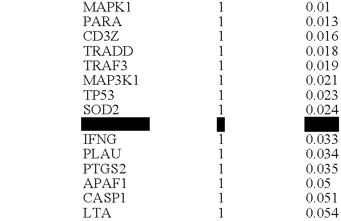

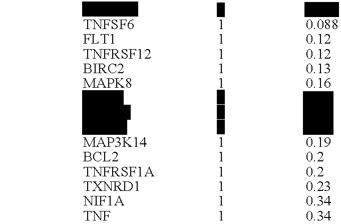

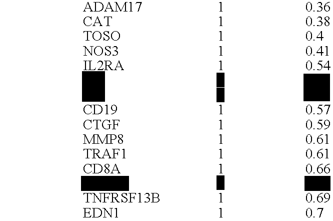

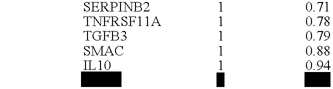

TABLE 13
1 and 2-gene POAG Models using MMP19 as the initial gene

| | 1 Gene | | | 2 Gene | |
|---|---|---|---|---|---|
| | % | | | % | % |
| | glaucoma | % Normal | | Glaucoma | Normal |
| Maximum = | 91% | 83% | Maximum = | 94% | 92% |
| Adjusted = | 85% | 92% | | | |
| TGFB1 | | | TGFB1 | | |
| | | | CD69 | | |

TABLE 14
Data for combined NPG and POAG Discrimination Line

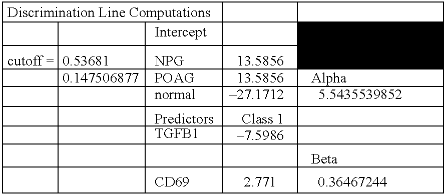

1. A method for determining a profile data set for characterizing a subject with ocular disease or a condition related to ocular disease, based on a sample from the subject, the sample providing a source of RNAs, the method comprising:
a) using amplification for measuring the amount of RNA in a panel of constituents including at least 1 constituent from Table 1A, Table 1B or Table 2, and
b) arriving at a measure of each constituent,
wherein the profile data set comprises the measure of each constituent of the panel and wherein amplification is performed under measurement conditions that are substantially repeatable.

2. A method of characterizing ocular disease or a condition related to ocular disease in a subject, based on a sample from the subject, the sample providing a source of RNAs, the method comprising:
assessing a profile data set of a plurality of members, each member being a quantitative measure of the amount of a distinct RNA constituent in a panel of constituents selected so that measurement of the constituents enables characterization of the presumptive signs of ocular disease, wherein such measure for each constituent is obtained under measurement conditions that are substantially repeatable.

3. The method of claim 2, wherein the panel comprises 69 or fewer constituents.

4. The method of claim 2, wherein the panel comprises 5 or fewer constituents.

5. The method of claim 2, wherein the panel comprises 2 constituents.

6. The method of claim 2, wherein the panel comprises 1 constituent.

7. A method of characterizing ocular disease according to claim 2, wherein the panel of constituents is selected so as to distinguish from a normal and an ocular disease-diagnosed subject.

8. The method of claim 7, wherein the panel of constituents distinguishes from a normal and an ocular disease-diagnosed subject with at least 75% accuracy.

9. A method of claim 2, wherein the panel of constituents is selected as to permit characterizing the severity of ocular disease in relation to a normal subject over time so as to track movement toward normal as a result of successful therapy.

10. The method of claim 2, wherein the panel includes TGFB1.

11. The method of claim 10, wherein the panel further includes one or more constituents selected from the group consisting of SERPINB2 and CD69.

12. The method of claim 2, wherein the panel includes MMP19.

13. The method of claim 12, wherein the panel further includes CD69.

14. A method of characterizing ocular disease or a condition related to ocular disease in a subject, based on a sample from the subject, the sample providing a source of RNAs, the method comprising:
determining a quantitative measure of the amount of at least one constituent of any constituent of Table 1A, Table 1B or Table 2 as a distinct RNA constituent, wherein such measure is obtained under measurement conditions that are substantially repeatable.

15. The method of claim 14, wherein the constituents distinguish from a normal and an ocular disease-diagnosed subject with at least 75% accuracy.

16. The method of claim 14, wherein said constituent is TGFB1, CRP, MADD, MMP19, CASP9, MMP13, NFKB1B, JUN, BCL3, BCL2L1, BAX, CD69, CD44, VDAC1, NFKB1, TIMP3, CD4, NOS2A, TRAF2, BIRC3, MMP2, MAPK14, IL8, HSPA1A, BIK, MMP9, MMP3, MMP12, PDCD8, C1QA, NOS1, TIMP1, TNFSF12, BID, ECE1, IL1RN, TNFRSF1B, TGFα, CD68, SAM, GSR, BAD, SERPINA3, BAK1, CD3Z, TRADD, MAPK1, PPARα, CASP3, TP53, TRAF3, MAP3K1, HLADRB1, SOD2, IFNG, PTGS2, PLAU, ANXA11, LTA, APAF1, CASP1, TOSO, CD19, MMP15, TNFRSF1A, BIRC2, GSTA1, PDCD8, and MMP1.

17. A method for predicting response to therapy in a subject having ocular disease or a condition related to ocular disease, based on a sample from the subject, the sample providing a source of RNAs, the method comprising:
a) determining a quantitative measure of the amount of at least one constituent of any constituent of Table 1A, Table 1B or Table 2 as a distinct RNA constituent, wherein such measure is obtained under measurement conditions that are substantially repeatable to produce patient data set; and
b) comparing the patient data set to a baseline profile data set, wherein the baseline profile data set is related to the ocular disease, or condition related to ocular disease.

18. A method for monitoring the progression of ocular disease or a condition related to ocular disease in a subject, based on a sample from the subject, the sample providing a source of RNAs, the method comprising:
a) determining a quantitative measure of the amount of at least one constituent of any constituent of Table 1A, Table 1B or Table 2 as a distinct RNA constituent in a sample obtained at a first period of time, wherein such measure is obtained under measurement conditions that are substantially repeatable to produce a first patient data set;
b) determining a quantitative measure of the amount of at least one constituent of any constituent of Table 1A, Table 1B or Table 2 as a distinct RNA constituent in a sample obtained at a second period of time, wherein such measure is obtained under measurement conditions that are substantially repeatable to produce a second profile data set; and
c) comparing the first profile data set and the second profile data set to a baseline profile data set, wherein the baseline profile data set is related to the ocular disease, or condition related to ocular disease.

19. A method for according to claim 2, wherein the measurement conditions that are substantially repeatable are within a degree of repeatability of better than ten percent.

20. The method of claim 2, wherein the measurement conditions that are substantially repeatable are within a degree of repeatability of better than five percent.

21. The method of claim 2, wherein the measurement conditions that are substantially repeatable are within a degree of repeatability of better than three percent.

22. The method of claim 2, wherein efficiencies of amplification for all constituents are substantially similar.

23. The method of claim 2, wherein the efficiency of amplification for all constituents is within ten percent.

24. The method of claim 2, wherein the efficiency of amplification for all constituents is within five percent.

25. The method of claim 2, wherein the efficiency of amplification for all constituents is within three percent.

26. The method of claim 2, wherein the sample is selected from the group consisting of blood, a blood fraction, body fluid, a population of cells and tissue from the subject.

27. The method of claim 2, wherein assessing further comprises:
comparing the profile data set to a baseline profile data set for the panel, wherein the baseline profile data set is related to the ocular disease, or condition related to ocular disease.

28. A kit for detecting ocular disease in a subject, comprising at least one reagent for the detection or quantification of any constituent measured according to claim 2 and instructions for using the kit.
